(19) United States
(12) Patent Application Publication
(10) Pub. No.: US 2010/0304373 A1
(43) Pub. Date: Dec. 2, 2010

(54) Methods for Assessing the Susceptibility of a Human to Diminished Health and Wellness

(76) Inventor: Robert P. Ricciardi, Kennet Square, PA (US)

Correspondence Address: Robert P. Ricciardi;Suite 200, 317 Wekiva Springs Road, Longwood, FL, 32779, US

(21) Appl. No.: 12/541,486

(22) Filed: Aug. 14, 2009

Related U.S. Application Data
(60) Provisional application No. 61/088,887, filed on Aug. 14, 2008.

(51) Int. Cl. C12Q 1/68

(52) U.S. Cl. 435/6

(57) ABSTRACT

The present invention relates to methods for assessing the advisability that a human should employ a compensatory composition comprised of one or more antioxidant ingredients. The methods involve assessing occurrence in the human's genome of a plurality of certain polymorphisms. Methods for determining the composition of preferred compensatory compositions are also disclosed.

### CROSS-REFERENCE TO RELATED APPLICATIONS

This application is entitled to priority pursuant to 35 U.S.C. § 119(e) to U.S. provisional patent application No. 61/088,887. The following co-pending applications and patents have common inventorship and/or common assignment: U.S. Pat. No. 7,211,383 for Kits and Methods for Assessing Skin Health; U.S. application Ser. No. 12/259,528 for Kits and Methods for Assessing Leptin-Mediated Lipid Metabolism; U.S. application Ser. No. 12/236,607 for Kits and Methods for Assessing the Coenzyme Q Reducing Status of a Patient, Including a Patient Ingesting a Statin; U.S. application Ser. No. 12/198,424 for Use of Genetic Information to Detect a Predisposition for Bone Density Conditions; U.S. application Ser. No. 11/931,447 for Kits and Methods for Assessing Oxidative Stress; and, U.S. application Ser. No. 11/731,180 for Kits and Methods for Assessing Skin Health. Each of the aforementioned applications are expressly incorporated herein.

### REFERENCE TO A MICROFICHE APPENDIX

Not applicable.

### BACKGROUND OF THE INVENTION

Nutritional genomics (coined “nutrigenomics”), is the science that studies how a person's diet or supplemented diet interacts with or as a function of his or her genotype to influence health and wellness. One of the main goals of nutrigenomics studies is to understand the relationship between a person's nutrition and his or her genetic predisposition to certain conditions. The present invention addresses the need for customized nutritional supplements taliored for an individual's genetic makeup to promote and preserve health. Nutrigenomics changes nutrition and nutritional supplementation from the subjective to the objective.

With the completion of the initial phase of the Human Genome Project, the stage has been set for the integration of genetics into nutritional supplements. The integration of genetics into nutrition science holds the promise of promoting and preserving healthy lifestyles. To that end, the present invention is a unique genomic testing method used to assess the assess the advisability that a human should employ a personalized nutritional supplementation system based on the assessment of the presence or absence of certain single nucleotide polymorphisms (“SNPs”) within the human's genome.

Basic nutritional supplementation is known in the art. Most basic nutritional supplements are mass-produced multi-vitamins consisting, primarily, of a mixture of vitamins and minerals which are intended to meet or exceed a recommended daily allowance (RDA) of the vitamins and minerals comprising the multi-vitamin. Other common basic nutritional supplements include compositions consisting of one or more key ingredients in composition with inactive filler ingredients or supplements intended for a specific purpose, such as increasing energy. A significant limitation to the efficacy of a basic nutritional supplement, such as those just described, is that they do not meet the specific nutritional needs of the individual taking them.

Extensive research over the last two decades, coupled with the sequencing of the human genome and the availability of public gene databases and state-of-the art technologies are leading to new links between genetic information and dietary factors. Such studies are the key to unlocking the details of how genetics and environmental exposures, including nutrition, interact.

Acquiring information about one's genome can be beneficial if it allows a person to take steps to protect and promote his or her health. With the passage of the Genetic Information Nondiscrimination Act, signed into law on May 21, 2008, individuals will be encouraged to learn their genetic make-up for the purpose of maximizing their nutrition, without worry of discrimination based on the details disclosed by assessment of their genetic information.

Most, if not all, human genes occur in a variety of forms which differ in at least minor ways. Heterogeneity in human genes is believed to have arisen, in part, from minor non-fatal mutations that have occurred in the human genome over time. In some instances, differences between alternative forms of a gene are manifested as differences in the amino acid sequence in a protein encoded by a gene. Some amino acid sequence differences can alter the reactivity or substrate specificity of the protein. Differences between the alternative forms of a gene can also affect the degree to which (if at all) the gene is expressed. However, many heterogeneties that occur in humans appear not to be correlated with any particular phenotype. Known heterogeneties include, for example, single nucleotide polymorphisms (“SNPs”) (i.e., alternative forms of a gene having a difference at a single nucleotide residue). Other known polymorphic forms include those in which sequences of larger portions (e.g., 2-1000 residues) of a gene exhibit numerous sequence differences and those which differ by the presence or absence of a portion of a gene.

Numerous disorders and physiological states have been associated with occurrence of one or more alternative forms of a gene in the genome of a human who exhibits a disease or physiological state. Accordingly, associations between individual disorders and individual polymorphisms are known. However, a need remains for a method of assessing a human's genome for overall health and wellness or predisposition for diminished health and wellness. Such assessments could be used to identify types and amounts of therapeutic, compensatory, inhibitory or preventive compositions or interventions that can be used to alleviate, inhibit, or prevent diminished health and wellness r promote vitality. This invention satisfies that need.

### BRIEF SUMMARY OF THE INVENTION

The invention relates to a method of assessing the health and wellness of a human, and more particularly, assessing the susceptibility of the human to diminished health and wellness such that the advisability that the human employ a compensatory ingredient can be determined. The method comprises assessing occurrence in the human's genome of at least 4 (and preferably six, eight, ten or each) of the panel of the claimed 12 polymorphisms, the panel consisting of:
a) the V16A polymorphism in the gene encoding MnSOD2 [rs4880];
b) the P198L polymorphism in the gene encoding GPX1 [rs1050450];
c) the Y113H polymorphism in the gene encoding mEPHX [rs1051740];
d) the Arg192Gln polymorphism in the gene encoding PON1 [rs662];
e) the C667T polymorphism in the gene encoding MTHFR [rs1801133];
f) the A66G, Ile22Met polymorphism in the gene encoding MTRR [rs1801394];
g) the A-308G polymorphism in the gene encoding TNFa [rs1800629];
h) the G/2G polymorphism in the gene encoding MMP-1 [rs1799750];
i) the T2M polymorphism in the gene encoding VDR [rs10735810];
j) the Pro187Ser polymorphism in the gene encoding NQO1 [rs1800566];
k) the C-344T polymorphism in the gene encoding CYP11B2 [rs1799998]; and,
l) the C2514T polymorphism in the gene encoding ApoB [rs693].

Additionally, adjunctive assessment of Sirtuin 1 (SIRT1) activity is also preferred. Studies have shown that SIRT1, which belongs to the silent information regulator 2, is involved in age-related pathologies. Zeng, L., et al., “Silent information regulator, Sirtuin 1, and age-related diseases” Geriatrics and Gerontology International, Volume 9, No. 1; March 2009, pp. 7-15(9). Such pathologies involve those which may be mediated by intervention with an antioxidant composition or other nutritional supplement and include cancer, metabolic diseases, cardiovascular disease, neurodegenerative conditions, osteoporosis, and chronic obstructive pulmonary disease.

The genes on which the aforementioned polymorphisms are assessed by the methods described herein are vitally important to overall health and specifically provide indication of a human's propensity for oxidative stress, environmental challenges, cardiovascular health, detoxification, immune health, neurological health, pulmonary health, vision health, and bone health. Occurrence of any of these polymorphisms (in the disadvantaged form described herein) is an indication that the human is predisposed to diminished health and wellness as compared to another human whose genome does not comprise the polymorphism, and whereby occurrence of a plurality of the polymorphisms (in the disadvantaged form as described herein) is an indication that the human exhibits an increased or enhanced susceptibility to diminished health and wellness as compared to a human whose genome does not comprise the plurality of these polymorphisms. Preferably, each of the 12 polymorphisms is assessed for purposes of determining susceptibility of the human to diminished health and wellness. Thereafter, it is preferable to assess for the presence or absence of the SIRT1 polymorphism in the human's genome. It is key to note that the effects of the presence of each of the aforementioned polymorphisms can be manipulated or mediated by nutritional supplementation.

In one embodiment, the method comprises assessing occurrence (either homozygous occurrence or heterozygous occurrence or absence) in the human's genome of at least 4 of the aforementioned polymorphisms. Adjunctive assessment of SIRT1 is also preferred. In other embodiments of the invention, the method comprises assessing occurrence (either homozygous occurrence or heterozygous occurrence or absence) of four, six, eight, ten, or each of these polymorphisms. Adjunctive assessment of SIRT1 is also preferred.

In another embodiment, substantially the same method can be used to assess the advisability that the human should employ a compensatory composition, such as a composition comprising antioxidants. When the method is used to assess the advisability that the human should employ a compensatory composition, occurrence of one or more of the polymorphisms in any of the 12 claimed polymorphisms (in the disadvantaged form as described herein) is an indication that that it is advisable for the human to employ or otherwise supplement his or her nutrition with a compensatory composition. This is also true relative to another human inasmuch as it is more advisable for a human whose genome comprises one or more of the 12 claimed polymorphisms to employ a compensatory composition or otherwise supplement his or her nutrition with a compensatory composition than another human whose genome does not comprise any of the polymorphisms assessed by the inventor's methods.

The method by which occurrence of the polymorphisms is assessed is not critical. For example, occurrence of the polymorphisms can be assessed using a method that includes contacting a nucleic acid derived from the human's genome with a first oligonucleotide. Use of an oligonucleotide has the advantage that it can be attached to a support using routine methods, and that a plurality of oligonucleotides can be attached to the same support to allow simultaneous detection of multiple polymorphisms. If a second oligonucleotide is used, then the allelic content of the human's genome can be determined. Detection of polymorphic sequences can be simplified by using labeled oligonucleotides, such as molecular beacon oligonucleotides.

Once the content of the human's genome for the polymorphisms selected for assessment has been determined, assessment of the human's susceptibility for diminished health and wellness can further comprise calculating a susceptibility score for the human. A susceptibility score can be calculated by summing, for each polymorphism assessed, the product of an assigned correlation factor. The correlation factor can be 2 for each instance of an advantaged allele and 1 for each instance of a disadvantaged allele.

In another aspect, the invention relates to a method of assessing the human's genome for the purpose of providing an instructional material advising the individual to employ a compensatory composition (namely, nutritional supplement, an antioxidant composition that exhibits antioxidant properties or a composition that can otherwise supplement the body's normal antioxidant mechanisms, such as alpha-lipoic acid or coenzyme Q or a customized compensatory composition). The instructional may further advise the individual on the benefits of the supplementation. This method comprises assessing occurrence in the human's genome for the 12 polymorphisms in the panel, as indicated above. Alternatively, this method may be achieved by assessment of four, six, eight or ten of the polymorphisms in the panel indicated above. Occurrence of any of the polymorphic forms (in the disadvantaged form as described herein, whether by one or two copies of the allele) is an indication that it is advisable that the human should employ a compensatory ingredient or composition. Occurrence of a plurality of polymorphisms (in the disadvantaged form as described herein, whether by one or two copies of the polymorphic allele) is an indication that it is even more advisable that the human should employ a compensatory ingredient or composition.

The inventor's methods are not directed to the formulation of any particular compensatory composition (i.e., a particular combination of ingredients). Rather, the inventor's methods are directed to advising an individual whose genome comprises one ore more disadvantaged alleles as determined by assessing the individual's genome for certain polymorphisms to employ a compensatory composition, nutritional supplement, or antioxidant. However, the methods disclosed herein can be used to formulate customized compensatory compositions intended to address, modulate, mediate or affect the expression of the key biochemical pathways measured by assessment of the SNPs contained in the panel.

### BRIEF DESCRIPTION OF THE SEVERAL DRAWINGS

The foregoing summary, as well as the following detailed description of the preferred embodiments of the invention, will be better understood when read in conjunction with the appended drawings. The invention is not limited to the instrumentality shown.

FIG. 1 depicts an example of results that can be obtained by analyzing occurrence of the assessed polymorphisms (contained within the panel). The results shown in FIG. 1 are for a hypothetical human that is:

a) Heterozygous for the V16A polymorphism in the gene encoding MnSOD2 [rs4880] (i.e., harbors one disadvantaged allele);

b) Homozygous for the the presence of the P198L polymorphism in the gene encoding GPX1 [rs1050450] (i.e., harbors two disadvantaged alleles);

c) Heterozygous for the Y113H polymorphism in the gene encoding mEPHX [rs1051740] (i.e., harbors one disadvantaged allele);

d) Homozygous for the absence of the Arg192Gln polymorphism in the gene encoding PON1 [rs662] (i.e., does not harbor any copies of the disadvantaged allele);

e) Homozygous for the absence of the C667T polymorphism in the gene encoding MTHFR [rs1801133] (i.e., does not harbor any copies of the disadvantaged allele);

f) Homozygous for the presence of the A66G, Ile22Met polymorphism in the gene encoding MTRR [rs1801394] (i.e., harbors two copies of the disadvantaged allele);

g) Homozygous for the absence of the A-308G polymorphism in the gene encoding TNFa [rs1800629] (i.e., does not harbor any copies of the disadvantaged allele);

h) Heterozygous for the G/2G polymorphism in the gene encoding MMP-1 [rs1799750] (i.e., harbors one disadvantaged allele);

i) Heterozygous for the T2M polymorphism in the gene encoding VDR [rs10735810] (i.e., harbors one disadvantaged allele);

j) Homozygous for the presence of the Pro187Ser polymorphism in the gene encoding NQO1 [rs1800566] (i.e., harbors two copies of the disadvantaged allele);

k) Homozygous for the absence of the C-344T polymorphism in the gene encoding CYP11B2 [rs1799998] (i.e., does not harbor any copies of the disadvantaged allele); and,

l) Heterozygous for the C2514T polymorphism in the gene encoding ApoB [rs693] (i.e., harbors one copy of the disadvantaged allele).

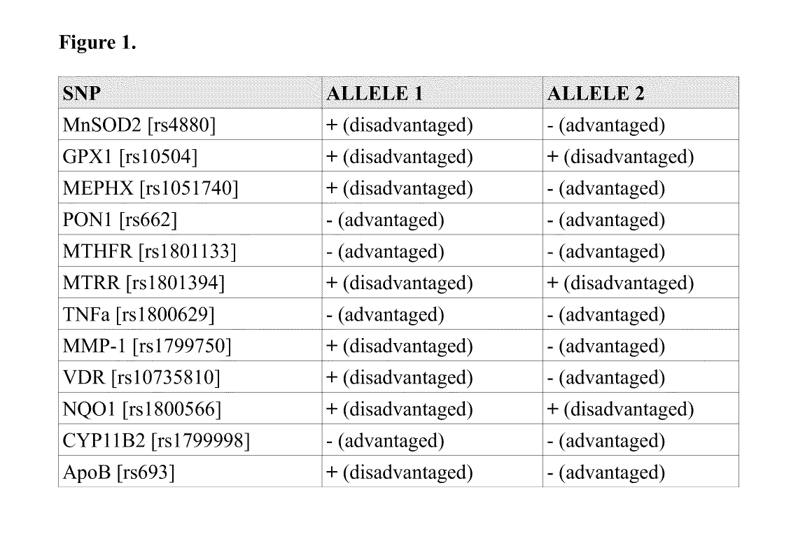

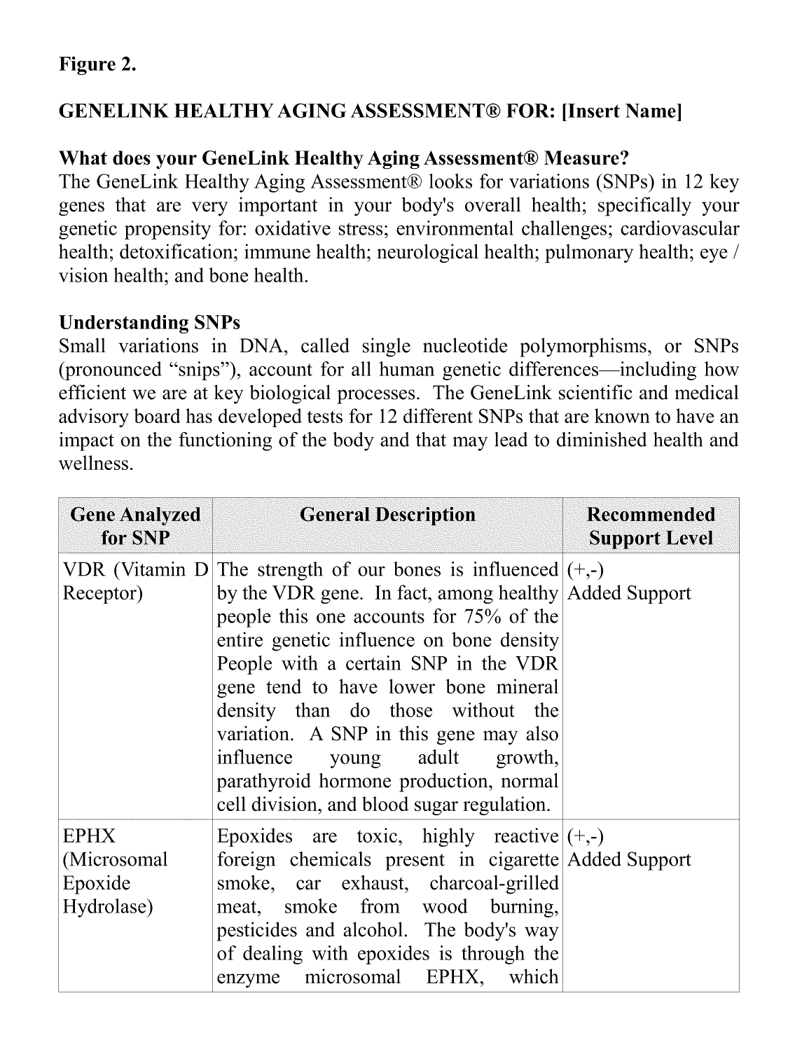

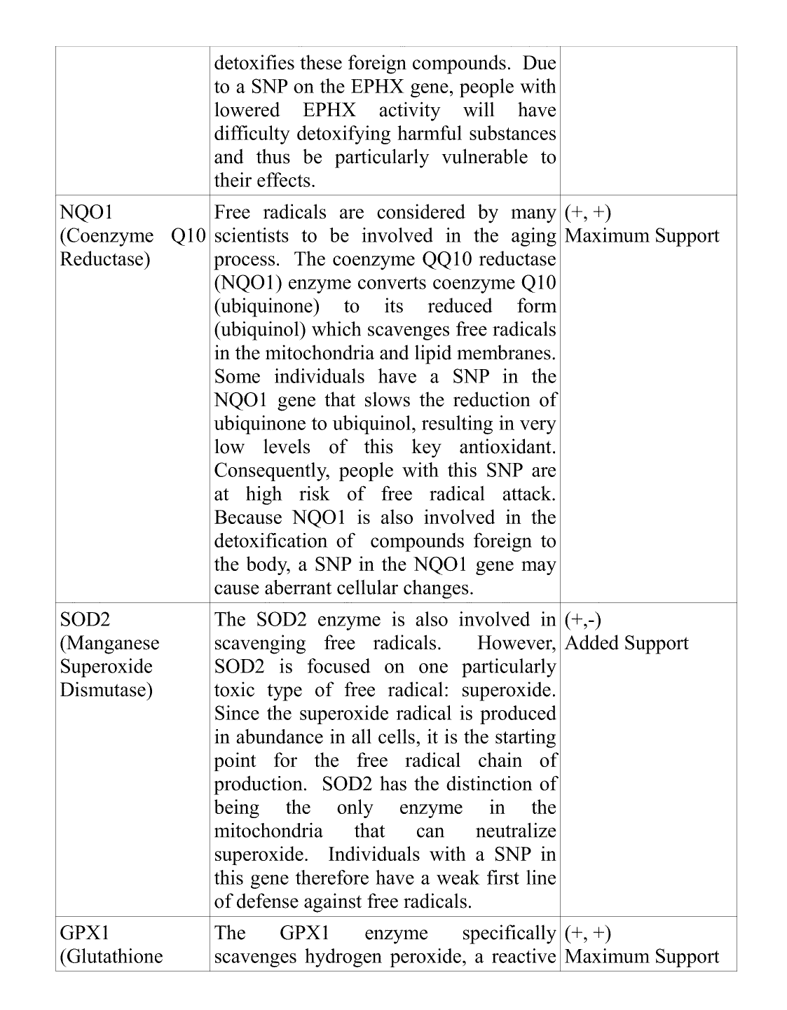

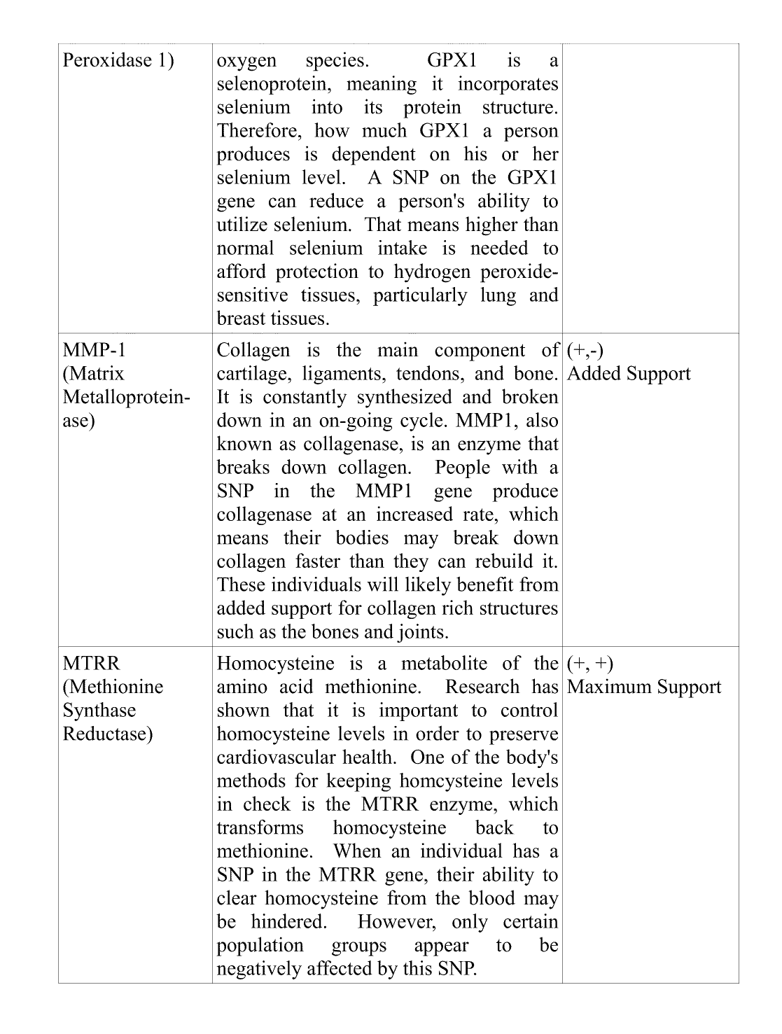

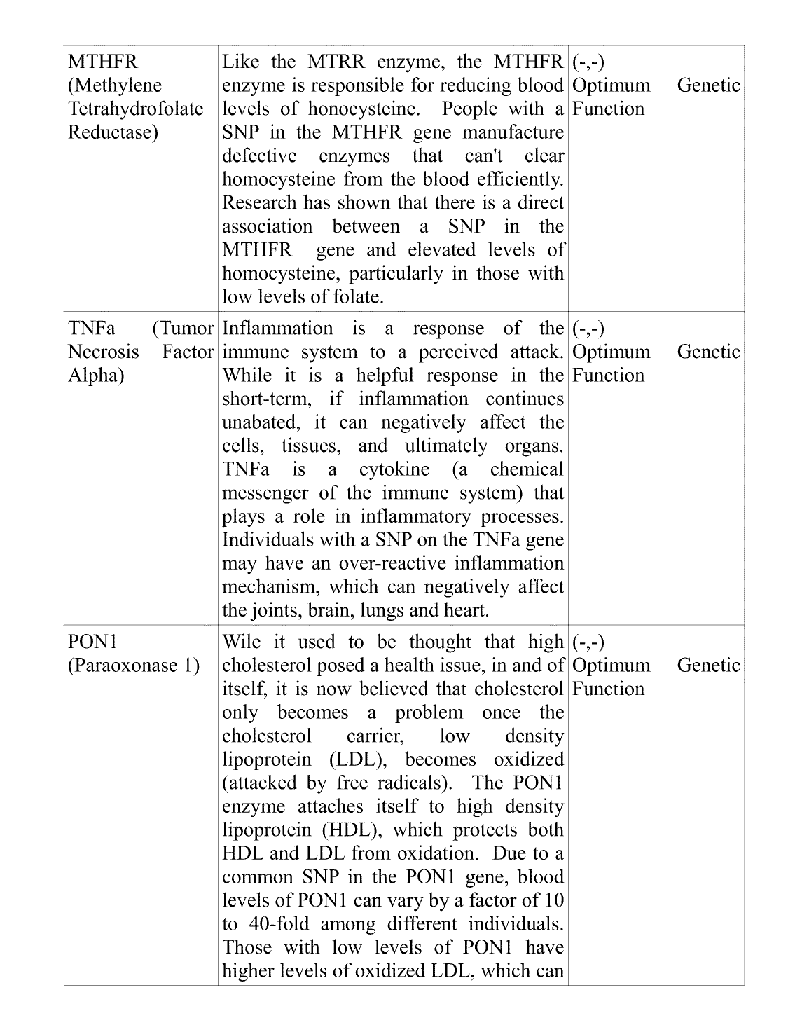

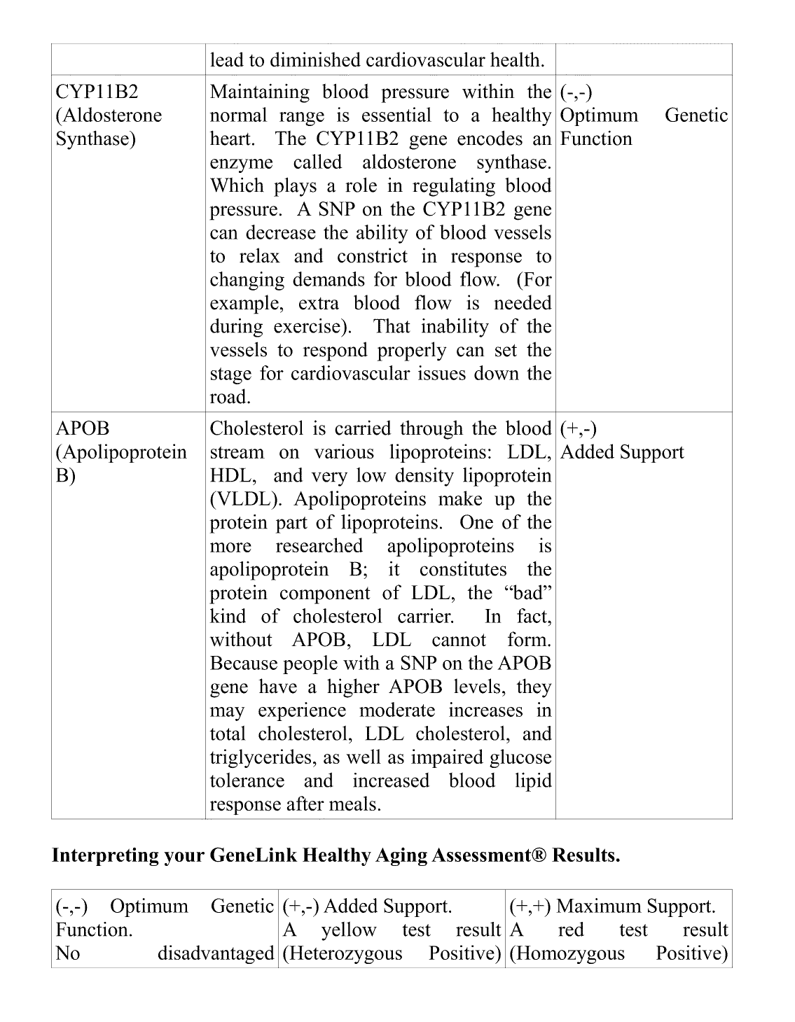

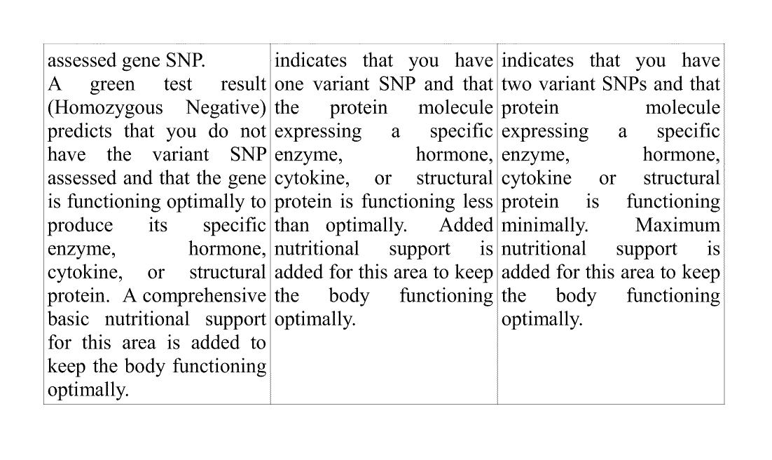

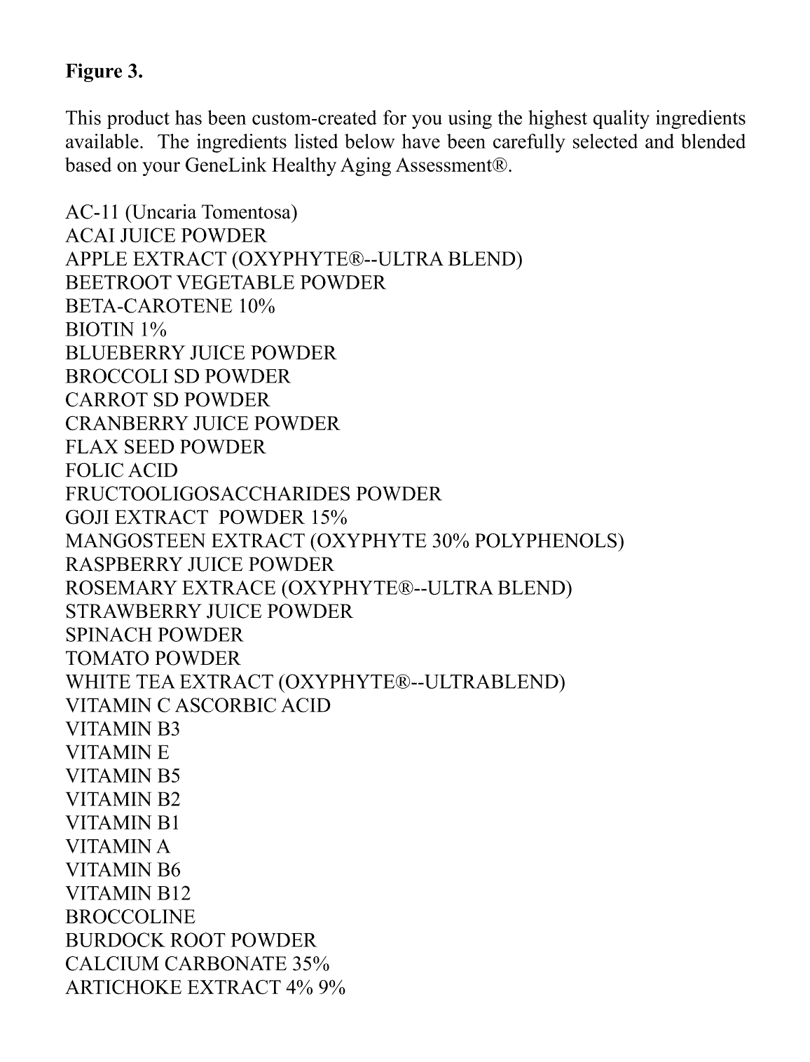

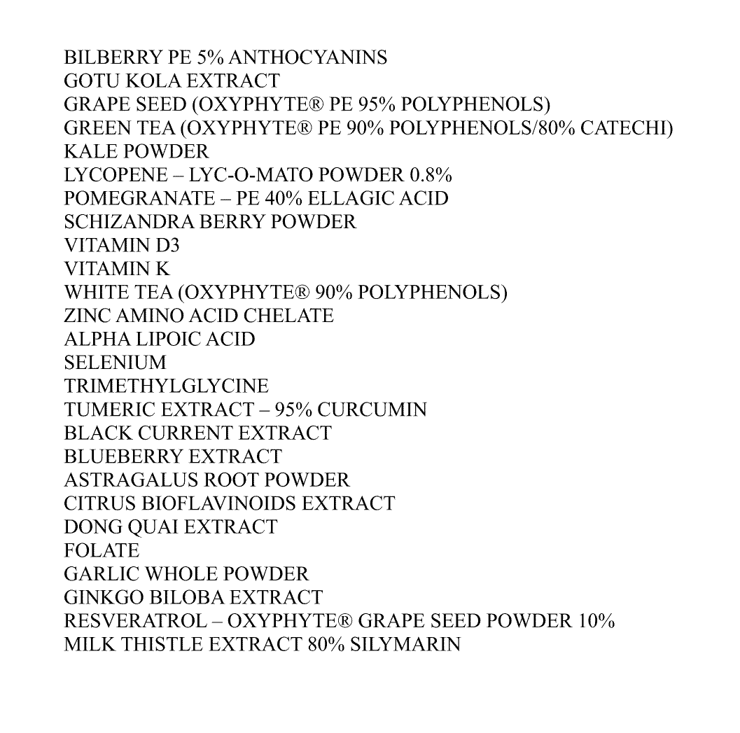

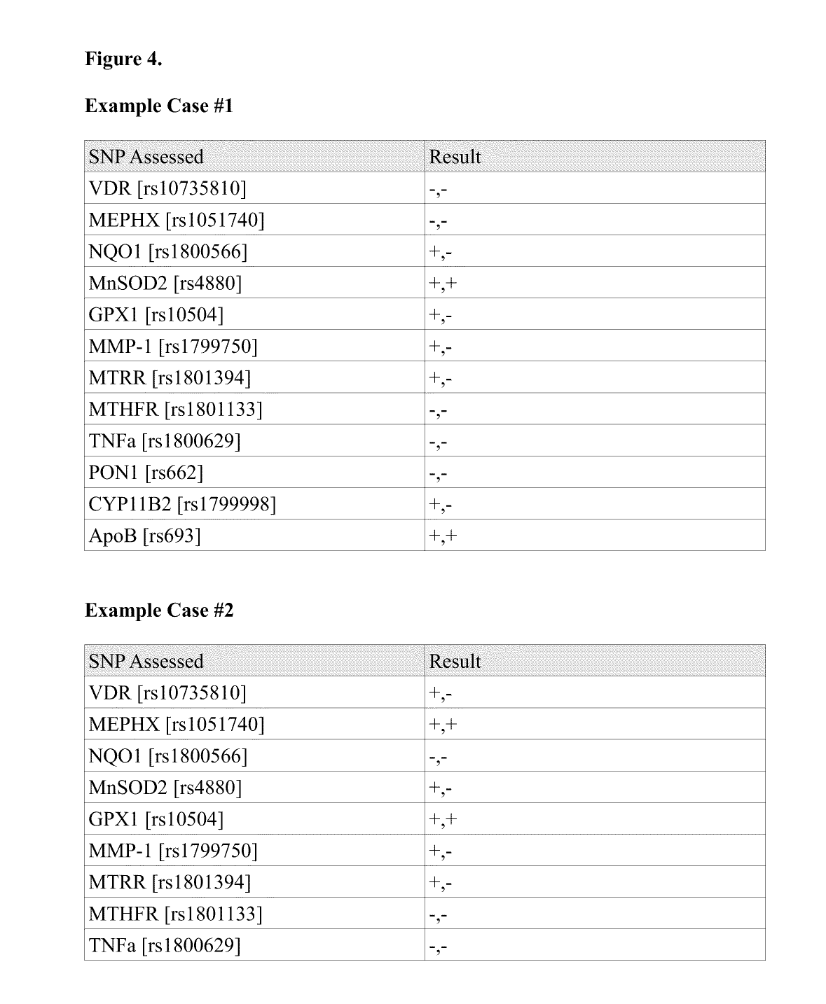

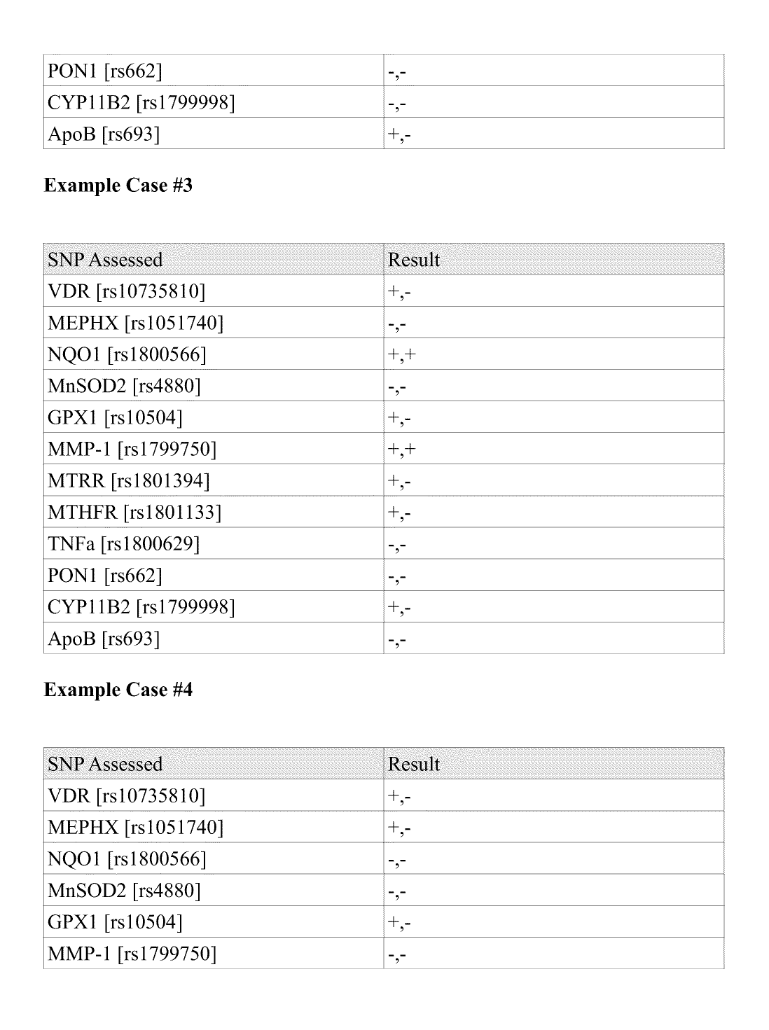

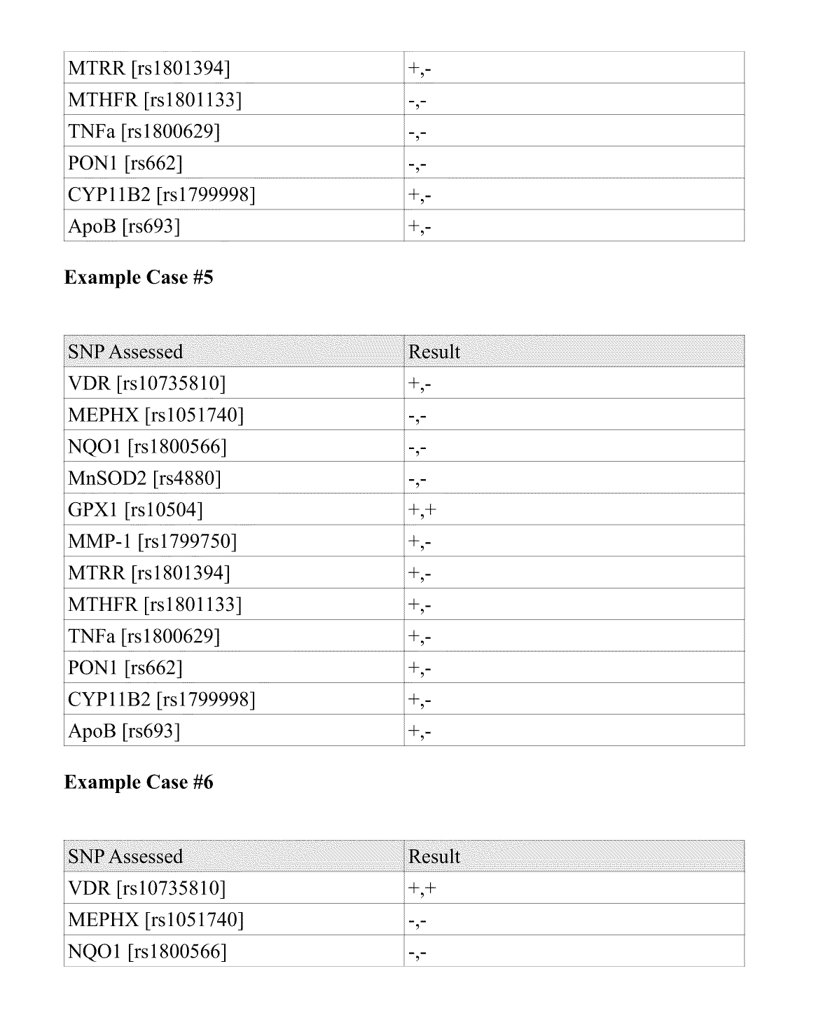

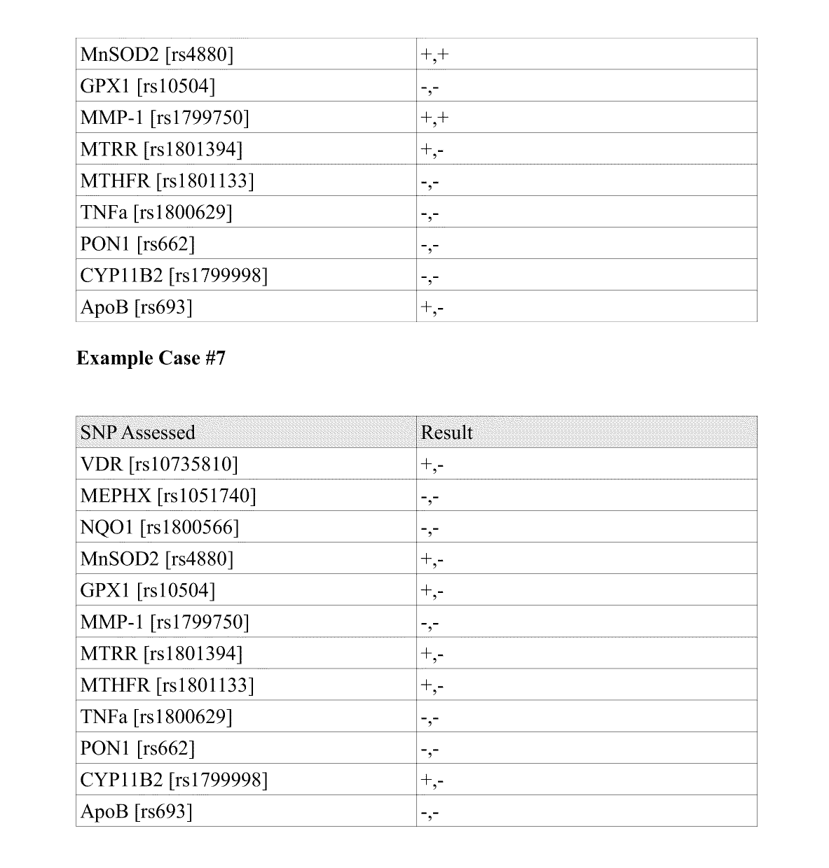

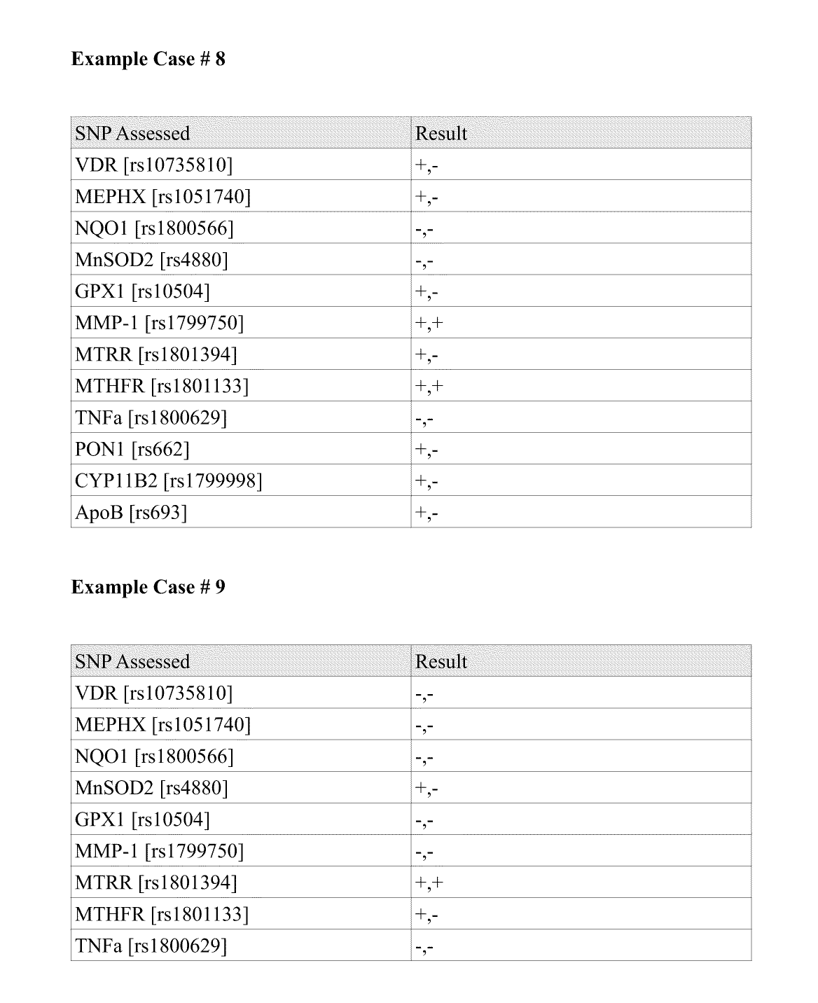

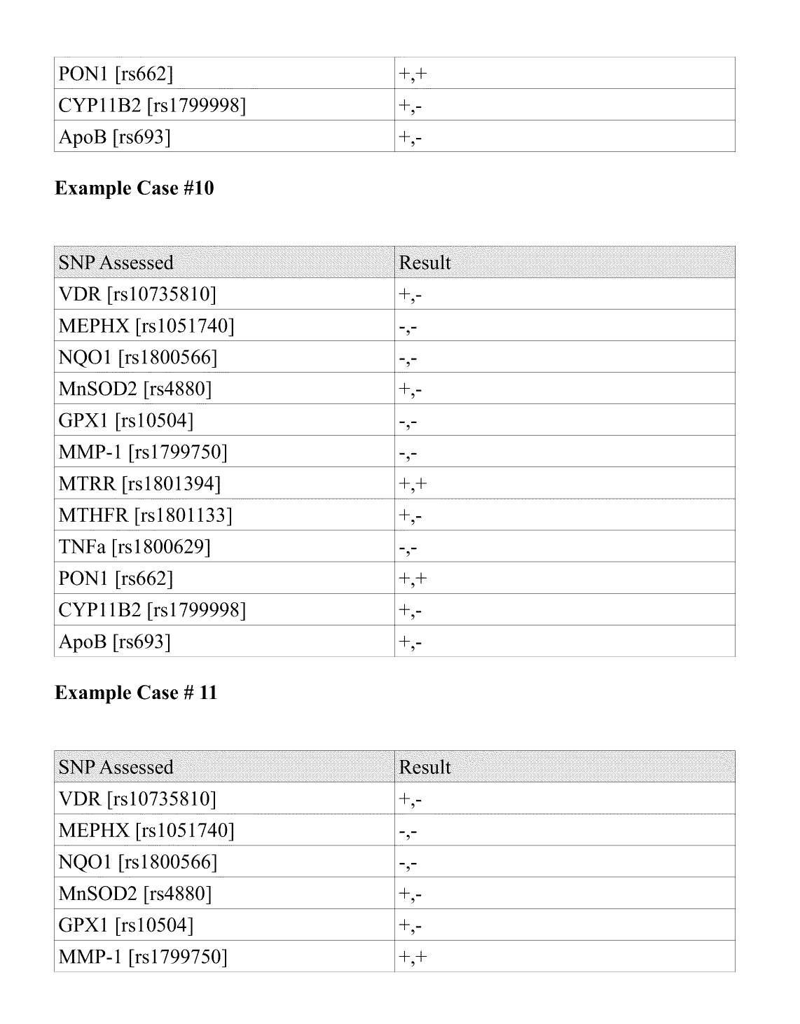

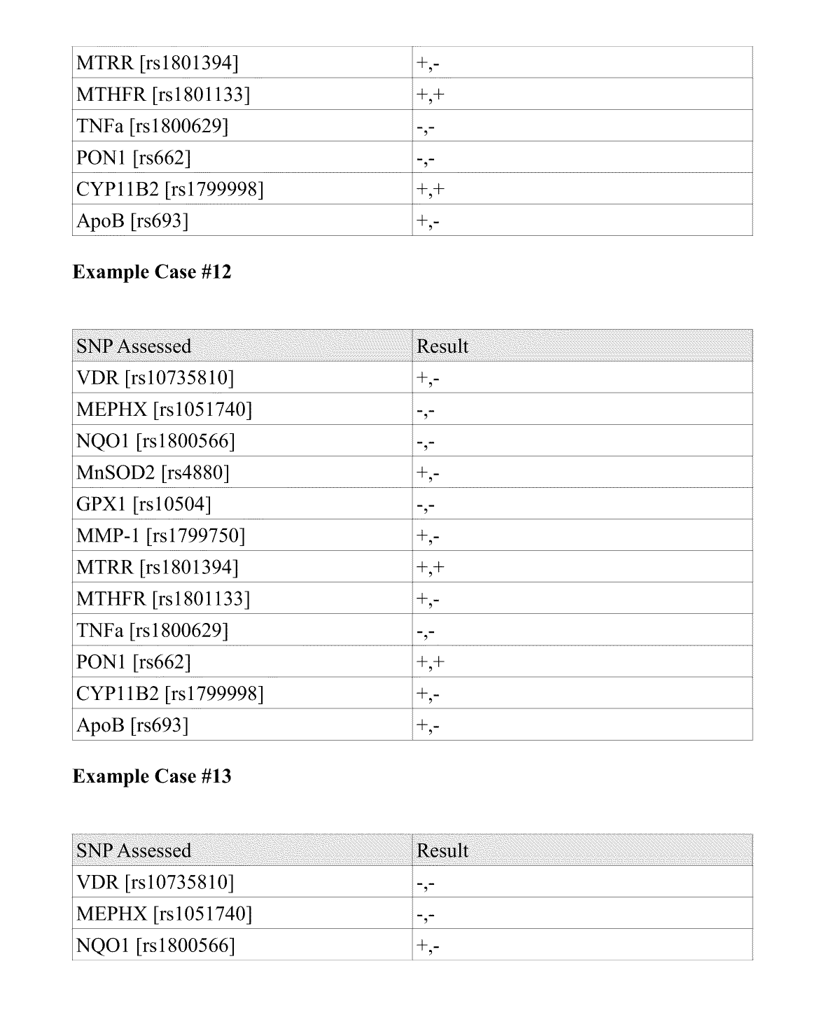

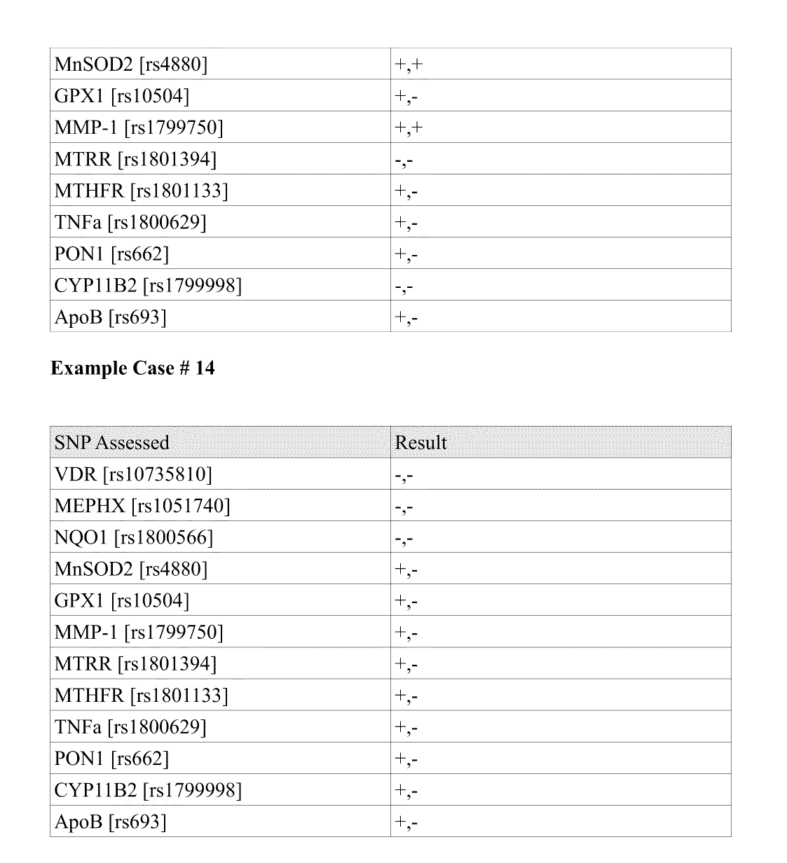

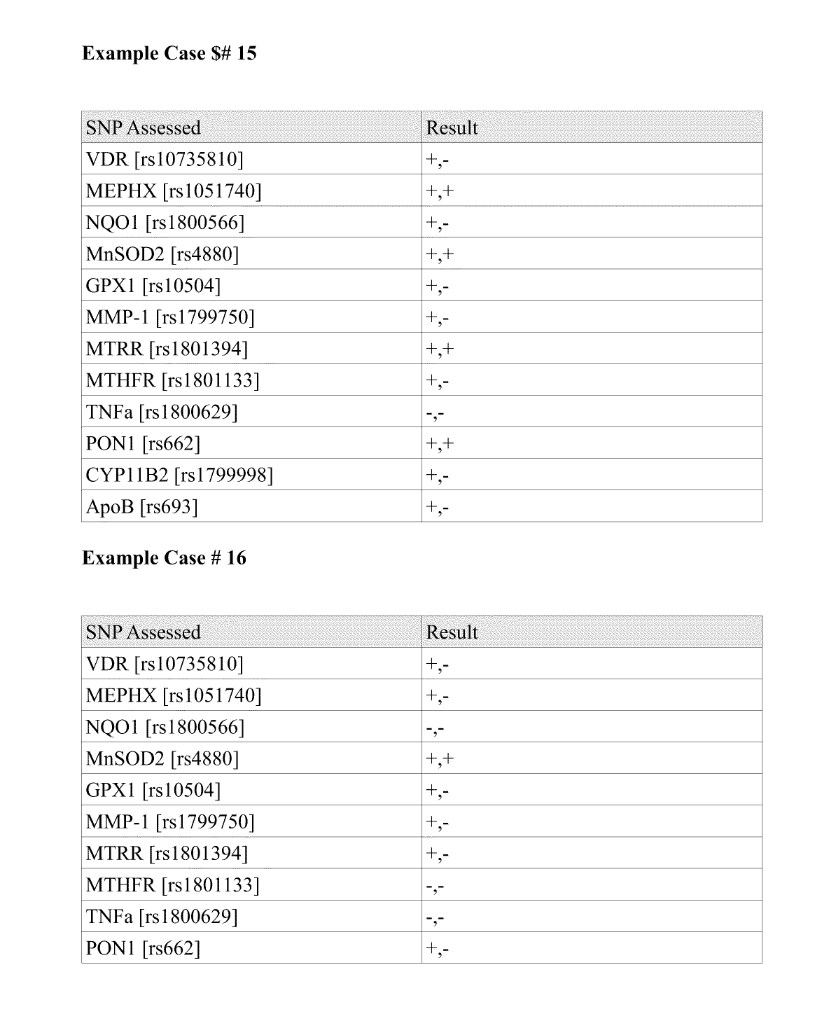

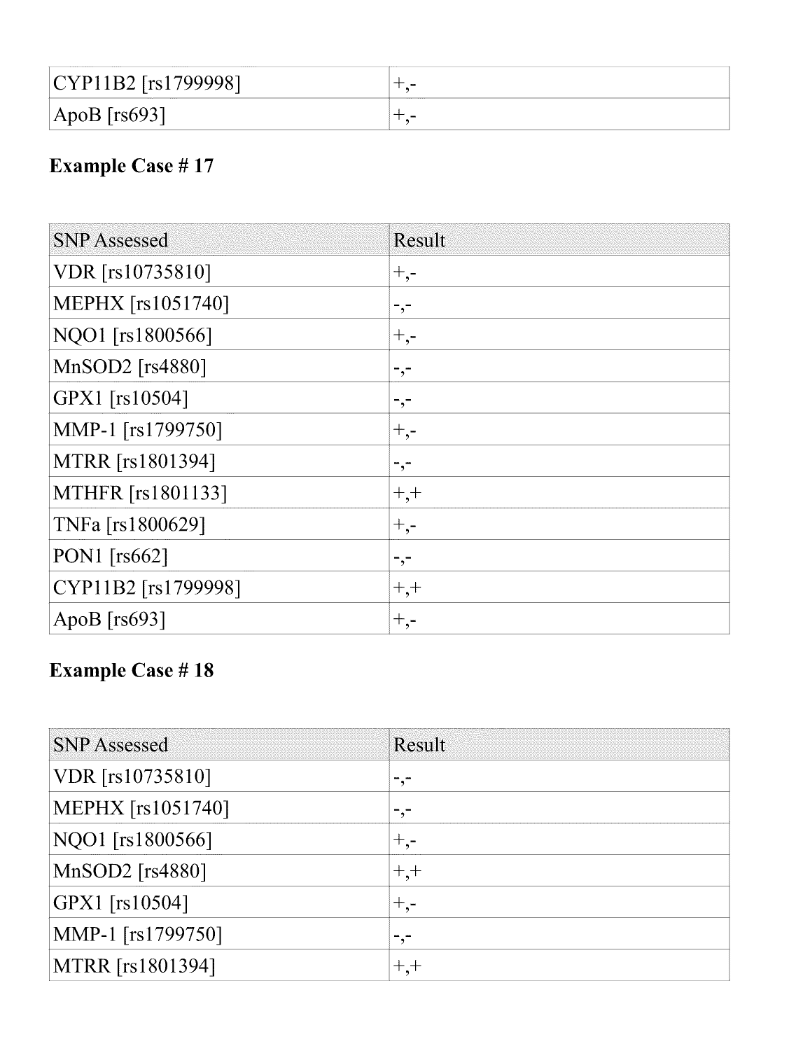

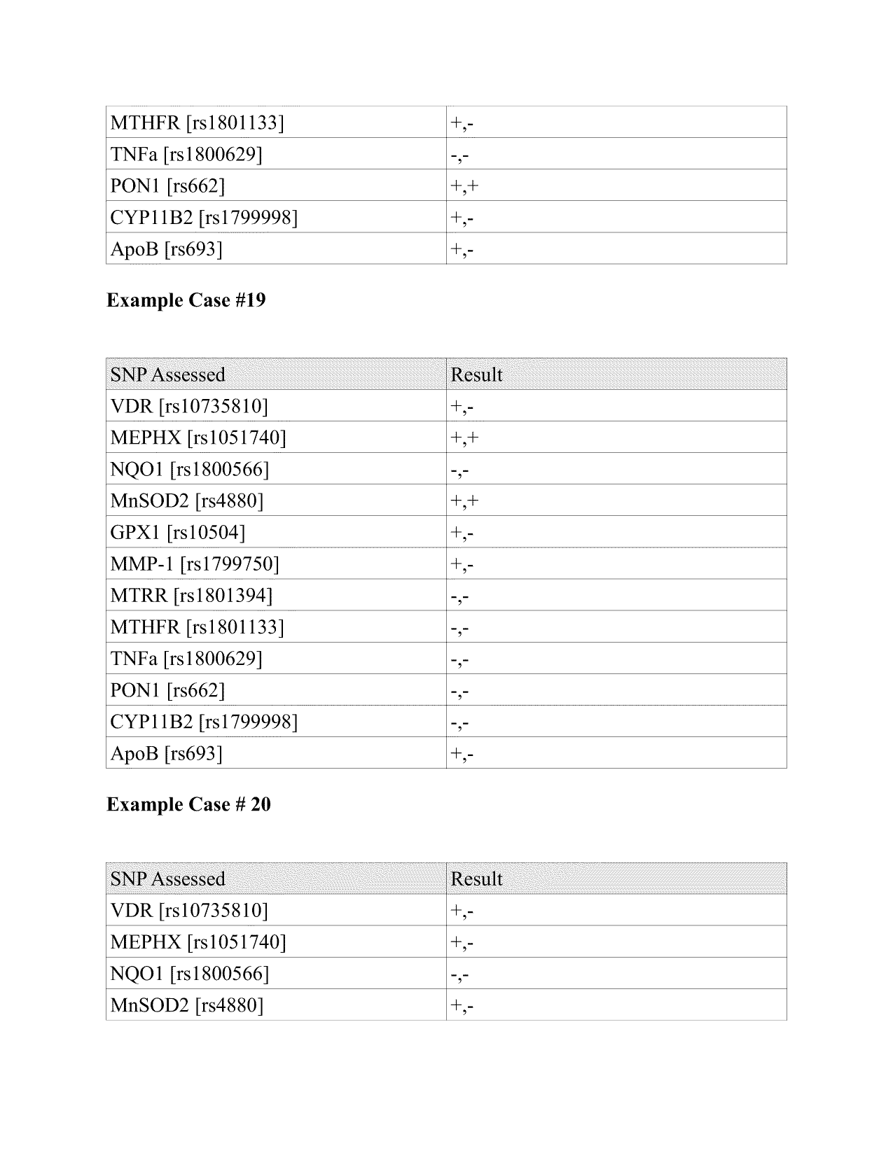

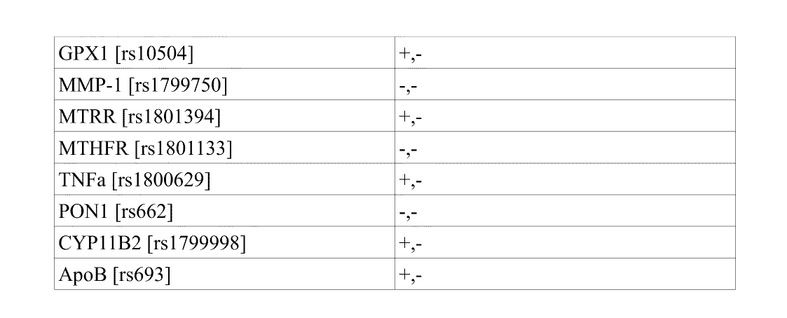

FIG. 2 is an image which depicts the instructional material that is prepared based on occurrence of the polymorphisms in the sample hypothetical human discussed in connection with FIG. 1.

FIG. 3 represents a potential compensatory composition for administration to the human discussed in FIGS. 1 and 2.

FIG. 4 represents twenty (20) working examples of the inventor's methods. These are actual examples of the methods disclosed herein and have been redacted for confidentiality purposes.

Each of the working examples illustrated in FIG. 4 (i) informs the individual of the presence or absence of disadvantaged alleles based on assessment of the SNPs assessed from the panel after a biological sample was obtained from the individual, which sample was assessed for each of the 12 polymorphisms in the panel; (ii) informs the individual whether he or she is advised to employ a nutritional supplement (or compensatory composition) in each example the individual is advised to employ a nutritional supplement or compensatory composition because each has at least one disadvantaged allele; and (iii) provides the individual with information on a recommended compensatory composition based on the assessment of the individual's genome for the presence or absence of the various SNPs assessed from the panel, this is a non-necessary step in the method disclosed herein. For each of the working examples (identified as “Example Cases”) presented in FIG. 4, the full panel of SNPs was assessed. However, the inventor's methods may be practiced by assessment of 4 or more SNPs from the panel. The biological samples which were assessed by this aforementioned method were collected using the collection kit described in U.S. Pat. No. 6,291,171 (Titled: Kit for the Non-Invasive Identification and Collection of DNA). Coincidentally, the working examples are in accordance with the best mode of practicing the invention set forth herein below.

### DETAILED DESCRIPTION OF THE INVENTION

The invention relates to methods for assessing the susceptibility of a human to diminished health and wellness and the advisability that the human should employ a nutritional supplement or compensatory composition by assessing occurrence in the human's genome of genetic polymorphisms. This assessment of health and wellness can be used to predict the likelihood that the human will develop, is predisposed to develop, or already has developed diminished health and wellness.

The methods involve determining whether multiple polymorphisms that have been associated (by the inventors or others) with human disorder (ie., disease or pathological state) occur in the genome of the human being assessed, thus providing an indication of the global state of health and wellness of the human. In some embodiments, the number of polymorphisms that occur in the human's genome are summed to yield a value; the higher the value is, the greater the susceptibility of the human to diminished health and wellness. The invention includes a variety of alternative methods and kits for performing the methods, as described herein in greater detail.

Each gene SNP assessed by the inventor's methods is relevant in the population. Presence of one or more copies of any of the SNPs (in the disadvantaged form described herein) assessed by the inventor's methods is a disadvantaged state and indicates that the individual is predisposed to diminished health and wellness. Based on the inventor's observations, the SNPs assessed by the methods disclosed herein occur in approximately the following frequencies.
At least one copy of the the V16A polymorphism in the gene encoding MnSOD2 [rs4880] appears in approximately 75 percent of the population;
At least one copy of the P198L polymorphism in the gene encoding GPX1 [rs1050450] appears in approximately 70 percent of the population;
At least one copy of the Y113H polymorphism in the gene encoding mEPHX [rs1051740] appears in approximately 35 percent of the population;
At least one copy of the Arg192Gln polymorphism in the gene encoding PON1 [rs662] appears in approximately 45 percent of the population;
At least one copy of the C667T polymorphism in the gene encoding MTHFR [rs1801133] appears in approximately 55 percent of the population;
At least one copy of the A66G, Ile22Met polymorphism in the gene encoding MTRR [rs1801394] appears in approximately 80 percent of the population;
At least one copy of the A-308G polymorphism in the gene encoding TNFa [rs1800629] appears in approximately 40 percent of the population;
At least one copy of the G/2G polymorphism in the gene encoding MMP-1 [rs1799750] appears in approximately 70 percent of the population;
At least one copy of the T2M polymorphism in the gene encoding VDR [rs10735810] appears in approximately 80 percent of the population;
At least one copy of the Pro187Ser polymorphism in the gene encoding NQO1 [rs1800566] appears in approximately 25 percent of the population;
At least one copy of the C-344T polymorphism in the gene encoding CYP11B2 [rs1799998] appears in approximately 80 percent of the population; and,
At least one copy of the C2514T polymorphism in the gene encoding ApoB [rs693] appears in approximately 85 percent of the population.

### Definitions

As used in this disclosure, the following terms have the meanings associated with them in this section.

A “polymorphism” is a gene in one of the alternative forms of a portion of a gene that are known to occur in the human population. For example, many genes are known to exhibit single nucleotide polymorphic forms, whereby the identity of a single nucleotide residue of a gene differs among the forms. Each of the polymorphic forms represents a single polymorphism, as the term is used herein. Other known polymorphic forms include alternative forms which multiple consecutive or closely-spaced, non-consecutive nucleotides vary in sequence, forms which differ by the presence or absence of a single nucleotide residue or a small number of nucleotide residues, also known respectively as insertion polymorphisms and deletion polymorphisms, and forms which exhibit different mRNA splicing patterns.

A “single nucleotide polymorphism” (“SNP”) is one of the alternative forms of a portion of a gene that vary only in the identity of a single nucleotide in that portion of the gene.

A “disorder-associated” polymorphism is an alternative form of a portion of a gene, wherein occurrence of the alternative form in the genome of the human has been correlated with exhibition by the human of a disease or pathological state, whether or not the disease or pathological state is exhibited by the human.

A “non-disorder-associated” polymorphism is an alternative form of a portion of a gene for which no significant positive correlation has been made between occurrence of the alternative portion of the gene in the genome and occurrence of a disease or pathological state. Non-disorder-associated polymorphisms are sometimes “neutral” polymorphisms in the art.

A disorder-associated polymorphism and a non-disorder-associated polymorphism “correspond” with one another if the two polymorphisms are two alternative forms of the same portion of the gene. By way of example, if the identity of residue 100 of a gene is an adenine in a disorder-associated polymorphism of the gene and cytosine in the non-disorder-associated polymorphism of the gene, them the two polymorphisms correspond to one another. It is understood that there are may be three or more corresponding polymorphisms when there are more than two alternative forms of the same portion of a gene.

A “characteristic residue” of a polymorphism is a nucleotide residue, the identity of which is known to vary among the alternative forms corresponding to the polymorphism.

“Health and wellness” is a measure of general well-being of an individual human. Health and wellness may be promoted by a healthy diet and appropriate nutritional supplementation. A great deal of literature in the art suggests that foods rich in antioxidants may have chemo protective properties and otherwise promote health and wellness. Accordingly, nutritional supplementation with a compensatory composition containing one or more antioxidant compounds may aid in maintenance of health and wellness in addition to promoting the same.

“Diminished health and wellness” is the presence of a pathological state in an individual human or the likelihood that the individual will develop, has a predisposition to develop, or is at risk of developing a disease or pathological state. Employing a compensatory composition (namely, a nutritional supplement or other intervention) may deter diminished health and wellness.

“Toxic oxygen species” include, in approximate order of decreasing reactivity, hydroxyl radicals, superoxide radicals, nitric oxide, peroxy nitrate (ONOO⁻; the product of reaction between nitric oxide and a superoxide radical), and hydrogen peroxide. Ordinary diatomic oxygen is not a toxic oxygen species, as the term is used herein.

“Oxidative damage” refers to a chemical reaction of a normal cellular component (e.g., DNA, a protein, or a lipid) with a toxic oxygen species, whereby at least one normal function of the component is inhibited or eliminated. As used herein, the terms “oxidative damage” and “oxidative stress” are used interchangeably.

A “molecular beacon oligonucleotide” is a single-stranded oligonucleotide having a fluorescent label (e.g., rhodamine, FAM, TET, VIC, JOE, or HEX) attached to the 5′-end thereof and a fluorescent quencher (e.g., TAMRA or DABCYL) attached to the 3′-end thereof (or vice versa), as described (Kostrikis et al., 1998, Science 279:1228 1229).

Two molecular beacon oligonucleotides are “spectrally distinct” if they can be differentially detected using spectrophotometric or spectroflurometric methods. Examples of characteristics that can be used to differentiate spectrally distinct oligonucleotides include absorption or excitation wavelength, emission wavelength, and fluorescent lifetime.

An “instructional material” is a publication, recording or diagram, or any other medium of expression which can be used to communicate how to use a method described herein. An instructional material may also communicate the results of practicing the methods described herein. Additionally, advisement to employ a compensatory composition, nutritional supplement, antioxidant, or other intervention may be contained in an instructional material. An instructional material, in any event, can be used cooperatively with the methods described herein.

The “stringency” with which two polynucleotides anneal means the relative likelihood that the polynucleotides will anneal in a solution as the conditions of the solution become less favorable for annealing. Examples of stringent conditions are known in the art and can be found in available references (e.g., Current Protocols in Molecular Biology, John Wiley & Sons, N.Y., 1989, 6.3.1-6.3.6). Aqueous and non-aqueous annealing methods are described in that reference and either can be used. In general, a first pair of oligonucleotides anneal with higher stringency than a second pair if the first pair is more likely to anneal (or remain annealed) as one or more of the slat concentration, temperature, and detergent concentration is increased.

With respect to a disorder, a “correlation factor” for disorder-associated polymorphisms is the fraction of humans who are heterozygous or homozygous for the polymorphism who exhibit the disorder. The correlation factor can, alternatively, be based solely on those who are heterozygous, solely on those who are homozygous, or on those who are either heterozygous or homozygous.

A “control” is an actual or hypothetical human whose genome does not comprise a single polymorphism assessed by the methods disclosed herein or an actual or hypothetical human whose genome comprises a known quantity of polymorphisms assessed by the methods disclosed herein.

A “non-extendable” nucleotide residue is a nucleotide residue that is capable of being added to a polynucleotide by a polymerase (i.e., by extension of the polynucleotide in association with a complement thereof, catalyzed by the polymerase) and that, upon addition to the polynucleotide, renders the polynucleotide incapable of being further extended by the polymerase.

A “reference SNP” or (“rs”) is a reported polymorphic variation, including summary information for the variation and a summary of allele frequencies as maintained by the National Center for Biotechnology Information (NCBI).

The “SNP panel” or “panel” as that term is used in this disclosure refers to those certain 12 SNPs assessed by the inventor's methods. Namely, a) the V16A polymorphism in the gene encoding MnSOD2 [rs4880]; b) the P198L polymorphism in the gene encoding GPX1 [rs1050450]; c) the Y113H polymorphism in the gene encoding mEPHX [rs1051740]; d) the Arg192Gln polymorphism in the gene encoding PON1 [rs662]; e) the C667T polymorphism in the gene encoding MTHFR [rs1801133]; f) the A66G, Ile22Met polymorphism in the gene encoding MTRR [rs1801394]; g) the A-308G polymorphism in the gene encoding TNFa [rs1800629]; h) the G/2G polymorphism in the gene encoding MMP-1 [rs1799750]; i) the T2M polymorphism in the gene encoding VDR [rs10735810]; j) the Pro187Ser polymorphism in the gene encoding NQO1 [rs1800566]; k) the C-344T polymorphism in the gene encoding CYP11B2 [rs1799998]; and, 1) the C2514T polymorphism in the gene encoding ApoB [rs693]. In some embodiments, less than the full panel of SNPs is assessed. However, in all embodiments, the SNPs assessed by the inventor's methods are selected from the aforementioned panel. The order in which the SNPs are assessed is not important.

A “disadvantaged” allele is characterized by the presence of at least one copy of the assessed SNP selected from the panel in the form which has been associated with a pathology or negative health consequence. Relative to each of the SNPs in the panel the disadvantaged alleles are as follows. In the V16A SNP in MnSOD2 [rs4880] the disadvantaged allele is the presence of a C at amino acid position 16; in the P198L SNP in GPX1 [rs1050450] the disadvantaged allele is the presence of a T at amino acid position 198; in the Y113H SNP in mEPHX [rs1051740] the disadvantaged allele is the presence of a C at amino acid position 113; in the Arg192Gln SNP in PON1 [rs662] the disadvantaged allele is the presence of a G at amino acid position 192; in the C667T SNP in MTHFR [rs1801133] the disadvantaged allele is the presence of a T at amino acid position 222; in the A66G, Ile22Met SNP in MTRR [rs1801394] the disadvantaged allele is the presence of a A at amino acid position 22; in the A-308G SNP in TNFa [rs1800629] the disadvantaged allele is the presence of a A at amino acid position −308; in the G/2G SNP in MMP-1 [rs1799750] the disadvantaged allele is the presence of a G at amino acid position −1607; in the T2M SNP in VDR [rs10735810] the disadvantaged allele is the presence of a A in exon 2 producing a new translational start site such that the length of the variant vitamin D receptor protein is increased by 3 amino acids; in the Pro187Ser SNP in NQO1 [rs1800566] the disadvantaged allele is the presence of a T at amino acid residue 187; in the C-344T SNP in CYP11B2 [rs1799998] the disadvantaged allele is the presence of a T at amino acid residue −344; and, in the C2514T SNP in ApoB [rs693] the disadvantaged allele is the presence of a T at amino acid position 2514.

A “compensatory composition” is a composition containing one or more antioxidants. A compensatory composition can be custom formulated based on the results of assessment of the SNPs in the panel such that the expression of the key biochemical pathways measured by assessment of the SNPs contained in the panel is mediated, modulated or affected by the compensatory composition.

An “intervention” is an advisement to employ or the actual employ of measures to improve health and wellness. Such measures can include use of a compensatory composition or nutritional supplement comprised of at least one antioxidant. Determination that the individual is predisposed to diminished health and wellness can also be used as an indication that the individual should be monitored closely for the development of certain disorders or pathological states as a form of intervention.

### BEST MODE OF PRACTICING THE INVENTION

The methods relating to assessing the advisability that a human should employ a compensatory composition is best practiced by: (1) collecting a biological sample from the human; (2) assessing, in the human's genome, for presence or absence of the polymorphisms in the panel; (3) providing an instructional material relating to the results of the genomic assessment; (4) advising individuals whose genome comprises one or more of the assessed polymorphisms in the disadvantaged form to employ a compensatory composition or other nutritional supplement or other intervention.

Most preferably, the biological sample is collected using the kit described in U.S. Pat. No. 6,291,171 (Titled: Kit for the Non-Invasive Identification and Collection of DNA). Collection by this means allows the sample to be self-collected in the privacy of one's own home. The collected sample may thereafter be remitted directly to a laboratory by regular mail without risk of contamination and without risk of otherwise compromising the integrity of the sample.

Once the collected sample is received for assessment, the sample is assessed for a plurality of SNPs identified in the panel. At least four SNPs are assessed (and more preferably six, eight, ten are assessed; and most preferably each of the SNPs identified in the panel are assessed).

Once the allelic content of the human's genome is known with respect to the SNPs assessed from the panel, an instructional material is prepared advising the individual of the results of the genomic assessment preformed further to the methods described herein. An example of an instructional material in this regard is provided as FIG. 2.

Individuals whose genome comprises at least one copy of a disadvantaged allele, as determined by the methods described herein, are advised to employ a compensatory composition, which composition is comprised of one or more antioxidants. The compensatory composition may be customized to the results of the genomic assessment whereby the compensatory composition ultimately employed by the individual is intended to address the expression of the key biochemical pathways measured by assessment of the SNPs contained in the panel.

In another embodiment, the invention relates to methods of formulating a compensatory composition for administration to the human and is best practiced by: (1) obtaining a biological sample from the human; (2) assessing the human's genome for presence or absence of polymorphisms in the panel; (3) determining the compensatory ingredients to comprise the compensatory composition; (4) providing an instructional material relating to the results of the genome assessment; and (5) advising the individual to employ a compensatory composition.

Most preferably, the biological sample is collected using the kit described in U.S. Pat. No. 6,291,171 (Titled: Kit for the Non-Invasive Identification and Collection of DNA). Collection by this means allows the sample to be self-collected in the privacy of one's own home. The collected sample may thereafter be remitted directly to a laboratory by regular mail without risk of contamination and without risk of otherwise compromising the integrity of the sample.

Once the collected sample is received for assessment, the sample is assessed for a plurality of SNPs identified in the panel. At least four SNPs are assessed (and more preferably six, eight, ten are assessed; and most preferably each of the SNPs identified in the panel are assessed).

The compensatory composition is then formulated according to the results of the genomic assessment whereby the compensatory composition ultimately employed by the individual is intended to address the expression of the key biochemical pathways measured by assessment of the SNPs contained in the panel. A review of the literature with respect to each of the SNPs contained in the panel will reveal appropriate ingredients for inclusion in the compensatory composition.

Thereafter, the individual is advised to employ the compensatory composition directed by the preceding step.

### Description

The invention relates to methods for assessing susceptibility of a human to diminished health and wellness by assessing occurrence in the human's genome of genetic polymorphisms that are associated with disorders. It has been discovered that the degree to which a human is susceptible to diminished health and wellness can be assessed by determining which polymorphic forms of certain genes are present in the human's genome. The polymorphisms in the genes which are assessed are associated with various disorders. Assessment of disorder-associated polymorphisms in four or more (and preferably six, eight, ten, or, most preferably, all) of theses genes is predictive of the human's health and wellness and predisposition to diminished health and wellness. The greater number of these genes in which occurrence of disorder-associated polymorphisms is assessed, the greater the precision for the methods for predicting the human's health and wellness. Occurrence in the human's genome of other polymorphisms (e.g., ones known to be associated with occurrence of disease or pathologies of interest) can also be assessed concurrently or sequentially.

Polymorphisms have been identified in some, if not all, of the numerous genes that encode components of the human DNA repair system. Disorder-associated polymorphisms in these genes can be informative for the health and wellness of the human. Examples of these genes are given.

Genes in which disorder-associated polymorphisms occur which are useful for assessing the health and wellness of an individual are disclosed in the art. It has been discovered that the genes disclosed in the 12-SNP panel are of particular relevance to the overall health of an individual.

Polymorphisms in an individual's genome can be assessed in a variety of ways. For example, a simple test (e.g., primer extension, PCR amplification, or molecular beacon oligonucleotide-binding) is used to determine whether or not the polymorphism occurs in the human's genome. Alternatively, a test (i.e., using multiple probes or primers) is used to detect the number of copies of a polymorphism which are present in the human's genome.

If it is determined that the individual is predisposed to diminished health and wellness (by presence of at least one of the assessed polymorphisms within the human's genome), then the individual can be encouraged to make changes to improve their health and wellness. Such changes can include use of a compensatory composition or nutritional supplement comprised of at least one antioxidant. Determination that the individual is predisposed to diminished health and wellness can also be used as an indication that the individual should be monitored closely for the development of certain disorders or pathological states.

Early detection of a predisposition to develop a disorder can enable an individual (or the individual's physician) to take steps to delay, inhibit, alleviate (i.e., reduce the severity of), or even prevent the disorder. The appropriate steps for treating and preventing disorders are well known and include modifying diet, exercise, and intake of nutrients and pharmaceuticals. Palliative, therapeutic, and prophylactic methods are known for many disorders, and healthy living more generally. These steps can be undertaken once an individual's susceptibility for a condition is known. Thus, the methods described herein permit an individual's health and wellness to be promoted. The methods described herein also allow the aforementioned interventions to be made at an early stage of the disorder.

It was not previously appreciated that detection in a human's genome of a plurality (two or more) of the polymorphisms assessed by the methods described herein is indicative that the human exhibit poorer health and wellness and manifests a greater susceptibility to certain disorders. Previous studies are believed to have recognized only associated between a polymorphism in only individual genes and a particular disorder. The inventor believes that he is the first to describe methods for assessing a human susceptibility to diminished health and wellness based on occurrence in the human's genome of certain polymorphisms (i.e., those disclosed herein).

### Key Genes Assessed in the Disclosed Methods

### Manganese Superoxide Dismutase (MnSOD2)

The MnSOD2 enzyme is involved in scavenging free radicals. In particular, MnSOD2 is focused on one exceptionally toxic type of free radical: superoxide. Since the superoxide radical is produced in abundance in all cells, it is the starting point for the free radical chain of production. Manganese superoxide dismutase has the distinction of being the only enzyme in the mitochondria that can neutralize superoxide. Individuals with the V16A SNP in MnSOD2, therefore, have a weak first line of defense against free radicals.

### Glutathione Peroxidase 1 (GPX1)

The GPX1 antioxidant enzyme specifically scavenges hydrogen peroxide, a reactive oxygen species. GPX1 is a selenoprotein, meaning that it incorporates selenium into its protein structure. Therefore, the amount of GPX1 a person produces is dependent on his or her selenium level. The P198L SNP in the GPX1 gene can reduce a person's ability to utilize selenium. That means higher levels than normal selenium intake is needed to afford protection to hydrogen peroxide sensitive tissues, particularly lung and breast tissues.

Microsomal Epoxide Hydrolase (mEPHX)

Epoxides are highly toxic, reactive foreign chemical present in cigarette smoke, car exhaust, charcoal-grilled meat, smoke from burning wood, pesticides and alcohol. The body's way of dealing with epoxides is through the enzyme mEPHX, which detoxifies these foreign compounds. Due to the Y113H SNP in mEPHX, some people will exhibit lowered mEPHX activity and will have difficulty detoxifying harmful substances and thus be particularly vulnerable to their effects.

### Paraoxonase 1 (PON1)

While it used to be thought that high cholesterol posed a health risk, in and of itself, it is now believed that cholesterol only becomes a problem once the cholesterol carrier low-density lipoprotein (LDL) becomes oxidized (i.e., is attacked by free radicals). The PON1 enzyme attaches itself to high-density lipoprotein (HDL) which protects both HDL and LDL fro oxidation. Due to the Arg192Gln SNP on the PON1 gene, blood levels of PON1 can vary by a factor of 10 to 40-fold among different individuals. Those with low levels of PON1 have higher oxidized levels of LDL, which can lead to diminished cardiovascular health.

### Methylene Tetrahydrofolate Reductase (MTHFR)

The MTHFR gene is responsible for reducing blood levels of homocysteine. Individuals with the C677T SNP in the MTHFR gene manufacture defective enzymes that can't clear homocysteine from the blood efficiently. Research has shown that there is a direct association between this SNP on the MTHFR gene and elevated levels of homocysteine, particularly in those individuals with low levels of folate.

### Methionine Synthase Reductase (MTRR)

Homocysteine is a metabolite of the amino acid methionine. Research has shown that it is important to control homocysteine levels in order to preserve cardiovascular health. One of the body's methods for keeping homocysteine levels in check is the MTRR gene, which transforms homocysteine back to methionine. When an individual has the A666, Ile22Met SNP in the MTRR gene, their ability to clear homocysteine is impaired.

### Tumor Necrosis Factor Alpha (TNFa)

Inflammation is a response of the immune system to a perceived attack. While it is a helpful response in the short term, if inflammation continues unabated, it can negatively affect the cells, tissues, and ultimately the organs. TNFa is a cytokine (a chemical messenger of the immune system) that plays a role in the inflammatory process. Individuals with the A-308G SNP in TNFa may have an over-reactive inflammation mechanism, which can negatively affect the joints, brain, lungs, and heart.

### Matrix Metallproteinase (MMP1)

Collagen is the main component of cartilage, ligaments, tendons, and bone. It is constantly synthesized and broken down in an on-going cycle. MMP1 is known as collagenase and is an enzyme that breaks down collagen. Individuals with the G/2G SNP in MMP1 produce collagenase at an increased rate, which means that their bodies may break down collagen faster than they can rebuild it. These individuals would benefit from added support for collagen-rich structures such as the bones and joints.

### Vitamin D Receptor (VDR)

The strength of our bones is influenced by the VDR gene. In fact, among healthy people, this one gene accounts for 75% of the entire genetic influence on bone density. People with the T2M SNP in the VDR gene tend to have lower bone mineral density than those without this variation. SNPs in this gene may also influence young adult growth, parathyroid hormone production, normal cell division, and blood sugar regulation.

### Coenzyme Q10 Reductase (NQO1)

Free radicals are considered by many scientists to be involved in the aging process. The NQO1 enzyme converts coenzyme Q (ubiquinone) to its reduced form (ubiquinone), which scavenges free radicals in the mitochondria and lipid membranes. Some individuals may have a SNP on the NQO1 gene, known as the Pro187Ser SNP, that slows the two-electron reduction of ubiquinone to ubiquinol, resulting in very low blood levels of this very important antioxidant. Consequently, people with this SNP are at high risk of free radical attack. Because NQO1 is also involved in the detoxification of compounds foreign to the body, this SNP in the NQO1 gene may cause aberrant cellular changes.

### Aldosterone Synthase (CYP11B2)

Maintaining blood pressure within the normal range is essential to a healthy heart. The CYP11B2 gene encodes an enzyme called aldosterone synthase, which plays a role in regulating blood pressure. The C-355T SNP in the CYP11B2 gene can decrease the ability of blood vessels to relax and constrict in response to changing demands for blood flow (e.g., during exercise). That inability of the vessels to respond properly can set the stage for cardiovascular issues.

### Apolipoprotein B (ApoB)

Cholesterol is carried through the blood stream on various lipoproteins: low-density lipoproteins (LDL), high-density lipoproteins (HDL), and very low-density lipoproteins (VLDL). Apolipoproteins make up the protein part of the lipoproteins. One of the more researched apolipoproteins is ApoB; it constitutes the protein component of LDL, the “bad” kind of cholesterol carrier. In fact, without ApoB, LDL cannot form. Because people with a SNP on the ApoB gene at position 2514 manifested as a change from a cytosine to a thymine, have higher ApoB levels, they experience moderate increases in total cholesterol, LDL cholesterol, and triglycerides, as well as impaired glucose tolerance and increased blood lipid responses after meals.

### Polymorphisms Assessed in the Disclosed Methods

MnSOD2 Polymorphism (rs4880)

The V16A polymorphism in MnSOD2 is part of the codon coding the amino acid valine at amino acid position 16 of MnSOD2. This polymorphism is manifested as a change from a thymine (T) to a cytosine (C) at amino acid position 16 of MnSOD2 which gives rise to an alanine at this amino acid position.

The advantaged homozygous genotype is TT; the disadvantaged heterozygous genotype is CT; and, the disadvantaged homozygous genotype is CC.

In one embodiment of the invention, when a customized report (instructional material) is generated specific to this SNP for purposes of communicating the results of the genomic assessment performed in connection with the methods disclosed herein, those individuals whose genotype is TT (i.e., advantaged homozygous) will be identified as having optimal genetic function, which may be expressed in equivalent language, requiring only basic nutritional supplementation (i.e., supplementation in accordance with RDAs) relative to compounds known in the art to address or otherwise impact or mediate the expression of the key biochemical pathways measured by assessment of this SNP. Those individuals whose genotype is CT (i.e., heterozygous disadvantaged) will be advised to employ added nutritional supplementation to support the enzymatic function of this gene. And, individuals whose genotype is CC (i.e., disadvantaged homozygous) will be advised to employ maximum nutritional supplementation to support the enzymatic function of this gene. The information relating to basic supplementation-basic support/added supplementation-added support/maximum-maximum support can be readily conveyed visually in an instructional material.

GPX1 Polymorphism (rs1050450)

The P198L polymorphism (rs1050450) in GPX1 is manifested as a change from a cytosine (C) to a thymine (T) at amino acid position 198 of that gene resulting in a proline to leucine substitution at this amino acid position.

The advantaged homozygous genotype is CC; the disadvantaged heterozygous genotype is TC; and, the disadvantaged homozygous genotype is TT.

In one embodiment of the invention, when a customized report (instructional material) is generated specific to this SNP for purposes of communicating the results of the genomic assessment performed in connection with the methods disclosed herein, those individuals whose genotype is CC (i.e., advantaged homozygous) will be identified as having optimal genetic function, which may be expressed in equivalent language, requiring only basic nutritional supplementation (i.e., supplementation in accordance with RDAs) relative to compounds known in the art to address or otherwise impact or mediate the expression of the key biochemical pathways measured by assessment of this SNP. Those individuals whose genotype is TC (i.e., heterozygous disadvantaged) will be advised to employ added nutritional supplementation to support the enzymatic function of this gene. And, individuals whose genotype is TT (i.e., disadvantaged homozygous) will be advised to employ maximum nutritional supplementation to support the enzymatic function of this gene. The information relating to basic supplementation-basic support/added supplementation-added support/maximum-maximum support can be readily conveyed visually in an instructional material.

mEPHX Polymorphism (rs1051740)

The Y113H SNP, designated rs 1051740, in mEPHX is manifested as a change from a cytosine (C) to a thymine (T) at amino acid position 113 of that gene resulting in a tyrosine to a histidine substitution at this amino acid position.

The advantaged homozygous genotype is TT; the disadvantaged heterozygous genotype is CT; and, the disadvantaged homozygous genotype is CC.

In one embodiment of the invention, when a customized report (instructional material) is generated specific to this SNP for purposes of communicating the results of the genomic assessment performed in connection with the methods disclosed herein, those individuals whose genotype is TT (i.e., advantaged homozygous) will be identified as having optimal genetic function, which may be expressed in equivalent language, requiring only basic nutritional supplementation (i.e., supplementation in accordance with RDAs) relative to compounds known in the art to address or otherwise impact or mediate the expression of the key biochemical pathways measured by assessment of this SNP. Those individuals whose genotype is CT (i.e., heterozygous disadvantaged) will be advised to employ added nutritional supplementation to support the enzymatic function of this gene. And, individuals whose genotype is CC (i.e., disadvantaged homozygous) will be advised to employ maximum nutritional supplementation to support the enzymatic function of this gene. The information relating to basic supplementation-basic support/added supplementation-added support/maximum-maximum support can be readily conveyed visually in an instructional material.

PON1 Polymorphism (rs662)

The Arg192Gln polymorphism, designated rs662, in PON1 is manifested as a change from an alanine (A) to a glutamic acid (G) at amino acid residue position 192 resulting in a glutamine to arganine substitution at this amino acid position.

The advantaged homozygous genotype is AA; the disadvantaged heterozygous genotype is AG; and, the disadvantaged homozygous genotype is GG.

In one embodiment of the invention, when a customized report (instructional material) is generated specific to this SNP for purposes of communicating the results of the genomic assessment performed in connection with the methods disclosed herein, those individuals whose genotype is AA (i.e., advantaged homozygous) will be identified as having optimal genetic function, which may be expressed in equivalent language, requiring only basic nutritional supplementation (i.e., supplementation in accordance with RDAs) relative to compounds known in the art to address or otherwise impact or mediate the expression of the key biochemical pathways measured by assessment of this SNP. Those individuals whose genotype is AG (i.e., heterozygous disadvantaged) will be advised to employ added nutritional supplementation to support the enzymatic function of this gene. And, individuals whose genotype is GG (i.e., disadvantaged homozygous) will be advised to employ maximum nutritional supplementation to support the enzymatic function of this gene. The information relating to basic supplementation-basic support/added supplementation-added support/maximum-maximum support can be readily conveyed visually in an instructional material.

MTHFR Polymorphism (rs1801133)

The C677T polymorphism, designated rs1801133, in the MTHFR gene is manifested as a change from a cytosine (C) to a thymine (T) at amino acid position 222 resulting in an alanine to valine substitution at this amino acid position.

The advantaged homozygous genotype is CC; the disadvantaged heterozygous genotype is CT; and, the disadvantaged homozygous genotype is TT.

In one embodiment of the invention, when a customized report (instructional material) is generated specific to this SNP for purposes of communicating the results of the genomic assessment performed in connection with the methods disclosed herein, those individuals whose genotype is CC (i.e., advantaged homozygous) will be identified as having optimal genetic function, which may be expressed in equivalent language, requiring only basic nutritional supplementation (i.e., supplementation in accordance with RDAs) relative to compounds known in the art to address or otherwise impact or mediate the expression of the key biochemical pathways measured by assessment of this SNP. Those individuals whose genotype is TC (i.e., heterozygous disadvantaged) will be advised to employ added nutritional supplementation to support the enzymatic function of this gene. And, individuals whose genotype is TT (i.e., disadvantaged homozygous) will be advised to employ maximum nutritional supplementation to support the enzymatic function of this gene. The information relating to basic supplementation-basic support/added supplementation-added support/maximum-maximum support can be readily conveyed visually in an instructional material.

MTRR Polymorphism (rs1801394)

The A666,Ile22Met polymorphism, designated rs1801394, in MTRR is manifested as a change from an adenine (A) residue to a guanine (G) residue at amino acid position 22 resulting in a isoleucine to methionine substitution at this amino acid position.

The advantaged homozygous genotype is GG; the disadvantaged heterozygous genotype is GA; and, the disadvantaged homozygous genotype is AA.

In one embodiment of the invention, when a customized report (instructional material) is generated specific to this SNP for purposes of communicating the results of the genomic assessment performed in connection with the methods disclosed herein, those individuals whose genotype is GG (i.e., advantaged homozygous) will be identified as having optimal genetic function, which may be expressed in equivalent language, requiring only basic nutritional supplementation (i.e., supplementation in accordance with RDAs) relative to compounds known in the art to address or otherwise impact or mediate the expression of the key biochemical pathways measured by assessment of this SNP. Those individuals whose genotype is GA (i.e., heterozygous disadvantaged) will be advised to employ added nutritional supplementation to support the enzymatic function of this gene. And, individuals whose genotype is AA (i.e., disadvantaged homozygous) will be advised to employ maximum nutritional supplementation to support the enzymatic function of this gene. The information relating to basic supplementation-basic support/added supplementation-added support/maximum-maximum support can be readily conveyed visually in an instructional material.

TNFa Polymorphism (rs1800629)

The A-308G polymorphism, designated rs1800629, is manifested as a change from an adenine (A) to a guanine (G) at amino acid −308 of TNFa.

The advantaged homozygous genotype is GG; the disadvantaged heterozygous genotype is GA; and, the disadvantaged homozygous genotype is AA.

In one embodiment of the invention, when a customized report (instructional material) is generated specific to this SNP for purposes of communicating the results of the genomic assessment performed in connection with the methods disclosed herein, those individuals whose genotype is GG (i.e., advantaged homozygous) will be identified as having optimal genetic function, which may be expressed in equivalent language, requiring only basic nutritional supplementation (i.e., supplementation in accordance with RDAs) relative to compounds known in the art to address or otherwise impact or mediate the expression of the key biochemical pathways measured by assessment of this SNP. Those individuals whose genotype is GA (i.e., heterozygous disadvantaged) will be advised to employ added nutritional supplementation to support the enzymatic function of this gene. And, individuals whose genotype is AA (i.e., disadvantaged homozygous) will be advised to employ maximum nutritional supplementation to support the enzymatic function of this gene. The information relating to basic supplementation-basic support/added supplementation-added support/maximum-maximum support can be readily conveyed visually in an instructional material.

MMP1 Polymorphism (rs1799750)

The G/2G SNP, designated rs 1799750, in MMP1 is an insertion polymorphism manifested as the presence of a guanine residue at amino acid residue −1607.

The advantaged homozygous genotype is the absence of a G at amino acid residue −1607 (i.e., −/−); the disadvantaged heterozygous genotype is −/G; and, the disadvantaged homozygous genotype is GG.

In one embodiment of the invention, when a customized report (instructional material) is generated specific to this SNP for purposes of communicating the results of the genomic assessment performed in connection with the methods disclosed herein, those individuals whose genotype is −/− (i.e., advantaged homozygous) will be identified as having optimal genetic function, which may be expressed in equivalent language, requiring only basic nutritional supplementation (i.e., supplementation in accordance with RDAs) relative to compounds known in the art to address or otherwise impact or mediate the expression of the key biochemical pathways measured by assessment of this SNP. Those individuals whose genotype is −/G (i.e., heterozygous disadvantaged) will be advised to employ added nutritional supplementation to support the enzymatic function of this gene. And, individuals whose genotype is GG (i.e., disadvantaged homozygous) will be advised to employ maximum nutritional supplementation to support the enzymatic function of this gene. The information relating to basic supplementation-basic support/added supplementation-added support/maximum-maximum support can be readily conveyed visually in an instructional material.

VDR Polymorphism (rs10735810)

The T2M polymorphism, designated rs10735810 in VDR is manifested as a change from a guanine (G) nucleotide to an adenine (A) nucleotide in exon 2, producing a new translational start site such that the length of the variant vitamin D (1,25-dihydroxyvitamin D3) receptor (VDR) protein is increased by 3 amino acids.

The advantaged homozygous genotype is GG; the disadvantaged heterozygous genotype is GA; and, the disadvantaged homozygous genotype is AA.

In one embodiment of the invention, when a customized report (instructional material) is generated specific to this SNP for purposes of communicating the results of the genomic assessment performed in connection with the methods disclosed herein, those individuals whose genotype is GG (i.e., advantaged homozygous) will be identified as having optimal genetic function, which may be expressed in equivalent language, requiring only basic nutritional supplementation (i.e., supplementation in accordance with RDAs) relative to compounds known in the art to address or otherwise impact or mediate the expression of the key biochemical pathways measured by assessment of this SNP. Those individuals whose genotype is GA (i.e., heterozygous disadvantaged) will be advised to employ added nutritional supplementation to support the enzymatic function of this gene. And, individuals whose genotype is AA (i.e., disadvantaged homozygous) will be advised to employ maximum nutritional supplementation to support the enzymatic function of this gene. The information relating to basic supplementation-basic support/added supplementation-added support/maximum-maximum support can be readily conveyed visually in an instructional material.

NQO1 Polymorphism (rs1800566)

The Pro187Ser polymorphism, designated rs 1800566, in NQO1 is manifested as a change from a cytosine (C) to a thymine (T) residue at amino acid residue 187 of NADP(H):quinone dehydrogenase, quinine 1 (NQO1).

The advantaged homozygous genotype is CC; the disadvantaged heterozygous genotype is CT; and, the disadvantaged homozygous genotype is TT.

In one embodiment of the invention, when a customized report (instructional material) is generated specific to this SNP for purposes of communicating the results of the genomic assessment performed in connection with the methods disclosed herein, those individuals whose genotype is CC (i.e., advantaged homozygous) will be identified as having optimal genetic function, which may be expressed in equivalent language, requiring only basic nutritional supplementation (i.e., supplementation in accordance with RDAs) relative to compounds known in the art to address or otherwise impact or mediate the expression of the key biochemical pathways measured by assessment of this SNP. Those individuals whose genotype is CT (i.e., heterozygous disadvantaged) will be advised to employ added nutritional supplementation to support the enzymatic function of this gene. And, individuals whose genotype is TT (i.e., disadvantaged homozygous) will be advised to employ maximum nutritional supplementation to support the enzymatic function of this gene. The information relating to basic supplementation-basic support/added supplementation-added support/maximum-maximum support can be readily conveyed visually in an instructional material.

CYP11B2 Polymorphism (rs1799998)

The C-344T SNP in CYP11B2, designated rs1799998, is manifested as a change from a cytosine residue to a thymine residue at amino acid residue −344 of cytochrome P450, family 11, subfamily B, polypeptide 2.

The advantaged homozygous genotype is CC; the disadvantaged heterozygous genotype is CT; and, the disadvantaged homozygous genotype is TT.

In one embodiment of the invention, when a customized report (instructional material) is generated specific to this SNP for purposes of communicating the results of the genomic assessment performed in connection with the methods disclosed herein, those individuals whose genotype is CC (i.e., advantaged homozygous) will be identified as having optimal genetic function, which may be expressed in equivalent language, requiring only basic nutritional supplementation (i.e., supplementation in accordance with RDAs) relative to compounds known in the art to address or otherwise impact or mediate the expression of the key biochemical pathways measured by assessment of this SNP. Those individuals whose genotype is CT (i.e., heterozygous disadvantaged) will be advised to employ added nutritional supplementation to support the enzymatic function of this gene. And, individuals whose genotype is TT (i.e., disadvantaged homozygous) will be advised to employ maximum nutritional supplementation to support the enzymatic function of this gene. The information relating to basic supplementation-basic support/added supplementation-added support/maximum-maximum support can be readily conveyed visually in an instructional material.

APOB Polymorphism (rs693)

The polymorphism designated rs693 is manifested as a change from a cytosine (C) to a thymine (T) at position 2514 in APOB.

The advantaged homozygous genotype is CC; the disadvantaged heterozygous genotype is CT; and, the disadvantaged homozygous genotype is TT.

In one embodiment of the invention, when a customized report (instructional material) is generated specific to this SNP for purposes of communicating the results of the genomic assessment performed in connection with the methods disclosed herein, those individuals whose genotype is CC (i.e., advantaged homozygous) will be identified as having optimal genetic function, which may be expressed in equivalent language, requiring only basic nutritional supplementation (i.e., supplementation in accordance with RDAs) relative to compounds known in the art to address or otherwise impact or mediate the expression of the key biochemical pathways measured by assessment of this SNP. Those individuals whose genotype is CT (i.e., heterozygous disadvantaged) will be advised to employ added nutritional supplementation to support the enzymatic function of this gene. And, individuals whose genotype is TT (i.e., disadvantaged homozygous) will be advised to employ maximum nutritional supplementation to support the enzymatic function of this gene. The information relating to basic supplementation-basic support/added supplementation-added support/maximum-maximum support can be readily conveyed visually in an instructional material.

### Disorder Associations

Although disorder associations do not form the foundation of the invention or the relevance of the polymorphisms assessed by the inventor's methods, each of the aforementioned polymorphisms is identified as being associated with one or more disorders. Practicing the claimed invention allows one skilled in the art to globally assess an individual's susceptibility to diminished health and wellness such that the advisability that a compensatory composition or ingredient should be employed by the individual can be determined.

By way of example only, the polymorphisms assessed by practicing the inventor's methods have been associated by the inventor's and others with specific pathologies or increased risk for certain pathologies.

The A16V (rs4880) polymorphism in MnSOD2 has been associated with an increased risk for heart disease and an increased risk for malignant pleural mesothelioma. See Valenti, L., et al., (2004, J. Med. Genet. 41(12):946-50); Landi, S., et al., (2007, Int. J. Cancer 120(12):2739-2743).

The Pro198Leu (rs1050450) polymorphism in GPX1 has been associated with an increased risk for breast and lung cancer. See Ravn-Haren, G., et al., (2006 Carcinogenesis 27(4):820-825).

The Y113H (rs1051740) polymorphism in mEPX1 has been associated with an increased risk for lymphoproliferative disorders. See e.g., Hasset, C., et al., (1994, Hum. Molec. Genet. 3:421-428); McGlynn, K. A., et al., (1995, Proc. Nat. Acad. Sci. 92: 2384-2387); Sarmanova, J. et al., (2001, Hum. Molec. Genet. 10:1265-1273).

The Arg 192Gln (rs662) polymorphism in PON1 has been associated with an increased risk for coronary heart disease in certain populations. See Odawara, M., et al., (1997, J. Clin. Endocrinol. Metab. 82(7):2257-2260).

The C677T (rs1801133) polymorphism in MTHFR has been associated with an increased risk for meningioma and glioma. See Bethke, L., et al., (2008, Cancer Epidemol. Biomarkers Prey. 17(5): 1195-1202).

The A22G (rs1801394) polymorphism in MTRR has been associated with heightened risk for neural tube defects, Down Syndrome and meningioma. See e.g. Wilson, A., et al., (1999, Molec. Genet. Metab. 67(4):317-323); Hobbs, C. A., et al., (2000, Am. J. Hum. Genet. 67(3): 623-630); Bethke, L., et al., (2008 Cancer Epidemol. Biomarkers Prey. 17(5):1195-1202).

The A-308G (rs1800629) polymorphism in TNFa has been associated with increased risks for liver cancer, systematic lupus erythematosus, heart disease, non-Hodgkin's lymphoma, Crohn's disease, and asthma. See e.g., Jeng, J. E., et al., (2007, Neoplasia. 9(11):987-992); Antonicelli, R., et al., (2005, Coron. Art. Dis. 16(8)489-493); Bel Hagj Jrad, B., et al., (2007, Eur. J. Hematol. 78(2):117-122); Ferreria, A. C., et al., (2005, Inflamm. Bowel Dis. 11(4):331-339); Aoki, T., et al., (2006, J. Hum. Genet. 51(8):677-685); Wu, H., et al., (2007, Environ. Health Persp. 115(4): 616-622).

The G/2G (rs1799750) polymorphism in VDR has been associated with an increased risk for ovarian cancer, hyperthyroidism, and diminished bone mineral density. See Galina, L., et al., (2007, Cancer Epidemol. Biomarkers Prey. 16(12):2566-2571); Horst-Sikorska, W. (2008, Endocrine Abstracts 16:68).

The Pro187Ser (rs1800566) polymorphism in NQO1 has been associated with poor survival among breast cancer patients, susceptibility to chronic benzene poisoning, and increased risk for lung, bladder and colorectal cancers. See Fagerholm, R., et al., (2008 Nature Genet.); Sun, P., et al., (2007 Genetic Polymorphisms of MPO, NQO1, GSTP1, UGT1A6 associated with susceptibility of chronic beneze poisoning); Chao, C., et al., (2006, Cancer Epidemol. Biomarkers Prey. 15:979-987).

The C-344T (rs1799998) polymorphism in CYP11B2 has been associated with an increased risk for hypertension. See Connell, J., et al., (2008 Endocrine Rev.).

The rs693 polymorphism in ApoB has been associated with elevated low density lipoprotein levels. See Saxony, R., et al., (2007, Science 316(5829):1331-1336).

Accordingly, in light of the foregoing, determining whether is it advised that the individual employ a compensatory composition can result in a positive intervention having the practical effect of preserving the health and well being of the individual.

### Compensatory Ingredients

Arriving at identification of the compensatory ingredients for each individual genotype as determined by assessment of the SNPs disclosed by the present invention is a step-process encompassed in at least one preferred embodiment of the invention.

Step 1—The gene SNP assessed is validated. A valid gene SNP is (a) relevant to a physiological condition as determined by the inventors or others; (b) prevalent in the population; (c) scientifically supported by third party literature in more than one reference; and (d) has a biochemical pathway that is impacted or modulated by nutritional supplementation.

Step 2—The gene SNP is included in the assessment method. In the case of the present invention, the 12 gene 12 which are included in the panel have been determined valid by the inventor.

Step 3—The assessment methods disclosed herein are performed.

Step 4—The allelic content of the human's genome is determined by the assessment methods.

Step 5—Compensatory ingredient(s) which would impact or modulate the biochemical pathway(s) of disadvantaged SNPs present in the human's genome are identified as suitable ingredients for inclusion in the compensatory composition. In the event that a customized compensatory composition is not desired, a compensatory composition includes any composition which includes at least one antioxidant ingredient wherein the individual has at least one disadvantaged allele present in his or her genome as determined by assessment of the SNP panel.

Ingredients suitable for inclusion in a compensatory composition include, by way of example and not limitation:

| | |
|---|---|
| AC-11 (Uncaria Tomentosa) | Green Tea |
| Acai juice powder | Vitamin B6 |
| Alpha Lipoic Acid | Vitamin C Ascorbic Acid |
| Anthocyanins | Vitamin D3 |
| Apple extract | Vitamin E |
| Artichoke extract | Vitamin K |
| Astragalus Root Powder | White Tea Extract |
| Beetroot Powder | Zinc Amino Acid |
| Beta-Carotene | Hawthorne Berry Extract |
| Biotin | Kale Powder |
| Black Currant Extract | Lycopene |
| Blackberry Extract | Magnesium Citrate |
| Blueberry Extract | Miatake Mushroom Extract |
| Blueberry Juice Powder | Mangosteen Extract |
| Broccoli SD Powder | Milk Thistle Extract |
| Broccoline Powder | Pomegranate Extract Powder |
| Burdock Root Powder | Potassium Citrate |
| Calcium Carbonate | Raspberry Juice Powder |
| Carrot SD Powder | Reshi Mushroom Extract |
| Cherry Extract | Resveratrol |
| Choline Bitartrate | Rosemary Extract |
| Citrus Bioflavanoids Extract | Schizandra Berry Powder |
| Cranberry Extract | Selenium |
| Cranberry Juice Powder | Shitake Mushroom Extract |
| Dong Quai Extract | Silymarin |
| Ellagic Acid | Spinach Powder |
| Flax Seed Powder | Strawberry Juice Powder |
| Folate | Tomato Powder |
| Folic Acid | Trimethylglycine |
| Fructoseoligosaccharrides Powder | Tumeric Extract |
| Garlic Whole Powder | Ubiquinone |
| Ginko Bilbao Extract | Ubiquinol |
| Goji Extract Powder | Vitamin A |
| Gotu Kola Extract | Vitamin B1 |
| Grape Seed | Vitamin B2 |
| Grape Skin | Vitamin B3 |
| | Vitamin B5 |

It has been discovered that the susceptibility of a human to diminished health and wellness as determined by occurrence in the human's genome of certain genetic polymorphisms can be assessed further indicating the advisability that an individual should employ a nutritional supplement, compensatory composition or an antioxidant.

It will be appreciated by those skilled in the art that changes can be made to the preferred embodiments described above without departing from the broad inventive concept thereof

The disclosure of every patent, patent application, and publication cited herein is hereby incorporated herein in its entirety.

This invention is not limited to the particular embodiments disclosed, and includes modifications within the spirit and scope of the present invention as defined by the appended claims.

What is claimed is:

1. A method of assessing the advisability that a human should employ a compensatory composition, the method comprised of
a) obtaining a biological sample from the human
b) assessing occurrence in the human's genome of a plurality of polymorphisms wherein the plurality of polymorphisms consists of at least four of of
i) a polymorphism manifested as a change from a thymine residue to a cytosine residue at amino acid residue 16 of mitochondrial superoxide dismutase 2 (MnSOD2);
ii) a polymorphism manifested as a change from a cytosine residue to a thymine residue at amino acid residue 198 of glutamate peroxidase 1 (GPX1);
iii) a polymorphism manifested a change from a thymine residue to a cytosine residue at amino acid residue 113 in exon 3 of microsomal epoxide hydrolase 1 (mEPHX1);
iv) a polymorphism manifested as a change from an adenine residue to a guanine residue at amino acid residue 192 of paraoxonasel (PON1);
v) a polymorphism manifested as a change from a cytosine residue to a thymine residue at amino acid residue 222 of 5,10-methylenetetrahydrofolate reductase (MTHFR) resulting in an alanine to valine substitution;
vi) a polymorphism manifested as a change from an adenine residue to a guanine residue at amino acid residue 22 of methionine synthase reductase (MTRR);
vii) a polymorphism manifested as a change from an adenine residue to a guanine residue at amino acid residue −308 of tumor necrosis factor alpha (TNFa);
viii) a polymorphism manifested as the presence of a guanine at amino acid residue −1607 of matrix metallproteinase 1 (MMPI)
ix) a polymorphism manifested as a change from a guanine nucleotide to an adenine nucleotide in exon 2, producing a new translational start site such that the length of the variant vitamin d (1,25-dihydroxyvitamin D3) receptor (VDR) is increased by three amino acids;
x) a polymorphism manifested as a change from a cytosine residue to a thymine residue at amino acid residue 187 of NAD(P)H:dehydrogenase, quinine 1 (NQO1);
xi) a polymorphism manifested as a change from a cytosine residue to a thymine residue at amino acid residue −344 of cytochrome P450, family 11, subfamily B, polypeptide 2 (CYP11B2);
xii) a polymorphism manifested as a change from a cytosine residue to a thymine residue at amino acid residue 2514 of apolipoprotein B (ApoB); and
c) providing an instructional material to the human wherein a human whose genome contains one or more disadvantaged alleles as determined by assessment of the polymorphisms selected from the panel is advised to employ a compensatory composition comprised of one or more antioxidants.

2. The method of claim 1 wherein the plurality of polymorphisms consists of at least 6 polymorphisms from the panel.

3. The method of claim 1 wherein the plurality of polymorphisms consists of at least 8 polymorphisms from the panel.

4. The method of claim 1 wherein the plurality of polymorphisms consists of at least 10 polymorphisms from the panel.

5. The method of claim 1 wherein the plurality of polymorphisms consists of each of the polymorphisms from the panel.

6. The method of claim 1 wherein the compensatory composition is comprised of ingredients intended to address, modulate, mediate or affect the expression of the key biochemical pathways measured by assessment of the polymorphisms assessed from the panel.

7. The method of claim 1 wherein the compensatory composition provides the likely benefit of promoting the health and wellness of the human.
